SEQUENCE LISTING (80 sequences)

SEQ ID NO: 1 - protein, 118 aa, Artificial Sequence
  source: /note=“Description of Artificial Sequence Synthetic polypeptide”
QVQLQQSGASMKMSCRASGYSFTSYWIHWLKQRPDQGLEWIGYIDPATAYTESNQKFKDKAILTADRSSNTAFMYLNSLTSEDSAVYYCARESPRLRRGIYYYAMDYWGQGTSVTVSS

SEQ ID NO: 2 - protein, 23 aa, Artificial Sequence
  source: /note=“Description of Artificial Sequence Synthetic peptide”
QVQLQQSGASMKMSCRASGYSFT

SEQ ID NO: 3 - protein, 5 aa, Artificial Sequence
  source: /note=“Description of Artificial Sequence Synthetic peptide”
SYWIH

SEQ ID NO: 4 - protein, 14 aa, Artificial Sequence
  source: /note=“Description of Artificial Sequence Synthetic peptide”
WLKQRPDQGLEWIG

SEQ ID NO: 5 - protein, 17 aa, Artificial Sequence
  source: /note=“Description of Artificial Sequence Synthetic peptide”
YIDPATAYTESNQKFKD

SEQ ID NO: 6 - protein, 32 aa, Artificial Sequence
  source: /note=“Description of Artificial Sequence Synthetic polypeptide”
KAILTADRSSNTAFMYLNSLTSEDSAVYYCAR

SEQ ID NO: 7 - protein, 16 aa, Artificial Sequence
  source: /note=“Description of Artificial Sequence Synthetic peptide”
ESPRLRRGIYYYAMDY

SEQ ID NO: 8 - protein, 11 aa, Artificial Sequence
  source: /note=“Description of Artificial Sequence Synthetic peptide”
WGQGTSVTVSS

SEQ ID NO: 9 - protein, 110 aa, Artificial Sequence
  source: /note=“Description of Artificial Sequence Synthetic polypeptide”
QSVVTQPPSASGGPGQSLTISCTGTSSDVGGYNHVSWYQQHPGKAPKLMIYDVSKRPSGVPHRFSGSKSGNTASLTVSGLQAEDEAVYYCSSYAGSNNLVFGGGTKVTVL

SEQ ID NO: 10 - protein, 105 aa, Artificial Sequence
  source: /note=“Description of Artificial Sequence Synthetic polypeptide”
DIQMTQTPSSLSASVGDRVTITCRASQSISSFLNWYQQKPGKAPKLLIYAASNLQSGVPSRFSGRGSGTDFTLTISSLQPEDFAAYYCQQGYTTPLTFGQGTKLE

SEQ ID NO: 11 - protein, 108 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 12 - protein, 15 aa, Artificial Sequence
  source: /note=“Description of Artificial Sequence Synthetic peptide”
EPKSCDKTHTCPPCP

SEQ ID NO: 13 - protein, 12 aa, Artificial Sequence
  source: /note=“Description of Artificial Sequence Synthetic peptide”
ERKCCVECPPCP

SEQ ID NO: 14 - protein, 62 aa, Artificial Sequence
  source: /note=“Description of Artificial Sequence Synthetic polypeptide”
ELKTPLGDTTHTCPRCPEPKSCDTPPPCPRCPEPKSCDTPPPCPRCPEPKSCDTPPPCPRCP

SEQ ID NO: 15 - protein, 12 aa, Artificial Sequence
  source: /note=“Description of Artificial Sequence Synthetic peptide”
ESKYGPPCPSCP

SEQ ID NO: 16 - protein, 16 aa, Artificial Sequence
  source: /note=“Description of Artificial Sequence Synthetic peptide”
APQAKSSGSGSESKVD

SEQ ID NO: 17 - protein, 22 aa, Artificial Sequence
  source: /note=“Description of Artificial Sequence Synthetic peptide”
QSVVTQPPSASGGPGQSLTISC

SEQ ID NO: 18 - protein, 14 aa, Artificial Sequence
  source: /note=“Description of Artificial Sequence Synthetic peptide”
TGTSSDVGGYNHVS

SEQ ID NO: 19 - protein, 15 aa, Artificial Sequence
  source: /note=“Description of Artificial Sequence Synthetic peptide”
WYQQHPGKAPKLMIY

SEQ ID NO: 20 - protein, 7 aa, Artificial Sequence
  source: /note=“Description of Artificial Sequence Synthetic peptide”
DVSKRPS

SEQ ID NO: 21 - protein, 32 aa, Artificial Sequence
  source: /note=“Description of Artificial Sequence Synthetic polypeptide”
GVPHRFSGSKSGNTASLTVSGLQAEDEAVYYC

SEQ ID NO: 22 - protein, 11 aa, Artificial Sequence
  source: /note=“Description of Artificial Sequence Synthetic peptide”
SSYAGSNNLVF

SEQ ID NO: 23 - protein, 9 aa, Artificial Sequence
  source: /note=“Description of Artificial Sequence Synthetic peptide”
GGGTKVTVL

SEQ ID NO: 24 - protein, 23 aa, Artificial Sequence
  source: /note=“Description of Artificial Sequence Synthetic peptide”
DIQMTQTPSSLSASVGDRVTITC

SEQ ID NO: 25 - protein, 11 aa, Artificial Sequence
  source: /note=“Description of Artificial Sequence Synthetic peptide”
RASQSISSFLN

SEQ ID NO: 26 - protein, 15 aa, Artificial Sequence
  source: /note=“Description of Artificial Sequence Synthetic peptide”
WYQQKPGKAPKLLIY

SEQ ID NO: 27 - protein, 7 aa, Artificial Sequence
  source: /note=“Description of Artificial Sequence Synthetic peptide”
AASNLQS

SEQ ID NO: 28 - protein, 32 aa, Artificial Sequence
  source: /note=“Description of Artificial Sequence Synthetic polypeptide”
GVPSRFSGRGSGTDFTLTISSLQPEDFAAYYC

SEQ ID NO: 29 - protein, 10 aa, Artificial Sequence
  source: /note=“Description of Artificial Sequence Synthetic peptide”
QQGYTTPLTF

SEQ ID NO: 30 - protein, 7 aa, Artificial Sequence
  source: /note=“Description of Artificial Sequence Synthetic peptide”
GQGTKLE

SEQ ID NO: 31 - protein, 22 aa, Artificial Sequence
  source: /note=“Description of Artificial Sequence Synthetic peptide”
SSELTQDPAVSVALGQTVRITC

SEQ ID NO: 32 - protein, 11 aa, Artificial Sequence
  source: /note=“Description of Artificial Sequence Synthetic peptide”
QGDSLRSYYAS

SEQ ID NO: 33 - protein, 15 aa, Artificial Sequence
  source: /note=“Description of Artificial Sequence Synthetic peptide”
WYQQKPGQAPVLVIY

SEQ ID NO: 34 - protein, 7 aa, Artificial Sequence
  source: /note=“Description of Artificial Sequence Synthetic peptide”
GKNNRPS

SEQ ID NO: 35 - protein, 32 aa, Artificial Sequence
  source: /note=“Description of Artificial Sequence Synthetic polypeptide”
GIPDRFSGSSSGNTASLTITGAQAEDEADYYC

SEQ ID NO: 36 - protein, 12 aa, Artificial Sequence
  source: /note=“Description of Artificial Sequence Synthetic peptide”
NSRDSSGNHVVF

SEQ ID NO: 37 - protein, 9 aa, Artificial Sequence
  source: /note=“Description of Artificial Sequence Synthetic peptide”
GGGTKLTVL

SEQ ID NO: 38 - protein, 21 aa, Artificial Sequence
  source: /note=“Description of Artificial Sequence Synthetic peptide”
GSGEGRGSLLTCGDVEENPGP

SEQ ID NO: 39 - protein, 63 aa, Artificial Sequence
  source: /note=“Description of Artificial Sequence Synthetic polypeptide”
GSGSRVTELLYRMKRAETYCPRPLLAIHPTEARHKQKIVAPVKQLLNFDLLKLAGDVESNPGP

SEQ ID NO: 40 - protein, 19 aa, Artificial Sequence
  source: /note=“Description of Artificial Sequence Synthetic peptide”
LLCFLLLLLSGDVELNPGP

SEQ ID NO: 41 - protein, 20 aa, Artificial Sequence
  source: /note=“Description of Artificial Sequence Synthetic peptide”
HHFMFLLLLLAGDIELNPGP

SEQ ID NO: 42 - protein, 20 aa, Artificial Sequence
  source: /note=“Description of Artificial Sequence Synthetic peptide”
WFLVLLSFILSGDIEVNPGP

SEQ ID NO: 43 - protein, 20 aa, Artificial Sequence
  source: /note=“Description of Artificial Sequence Synthetic peptide”
KNCAMYMLLLSGDVETNPGP

SEQ ID NO: 44 - protein, 20 aa, Artificial Sequence
  source: /note=“Description of Artificial Sequence Synthetic peptide”
MVISQLMLKLAGDVEENPGP

SEQ ID NO: 45 - protein, 22 aa, Artificial Sequence
  source: /note=“Description of Artificial Sequence Synthetic peptide”
GSGATNFSLLKQAGDVEENPGP

SEQ ID NO: 46 - protein, 23 aa, Artificial Sequence
  source: /note=“Description of Artificial Sequence Synthetic peptide”
GSGQCTNYALLKLAGDVESNPGP

SEQ ID NO: 47 - DNA, 500 bp, Artificial Sequence
  source: /note=“Description of Artificial Sequence Synthetic polynucleotide”
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 48 - DNA, 123 bp, Artificial Sequence
  source: /note=“Description of Artificial Sequence Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 49 - DNA, 81 bp, Artificial Sequence
  source: /note=“Description of Artificial Sequence Synthetic oligonucleotide”
ttttgggtgctggtggtggttggtggagtcctggcttgctatagcttgctagtaacagtggcctttattattttctgggtg

SEQ ID NO: 50 - DNA, 339 bp, Artificial Sequence
  source: /note=“Description of Artificial Sequence Synthetic polynucleotide”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 51 - DNA, 127 bp, Artificial Sequence
  source: /note=“Description of Artificial Sequence Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcg

SEQ ID NO: 52 - protein, 27 aa, Artificial Sequence
  source: /note=“Description of Artificial Sequence Synthetic peptide”
FWVLVVVGGVLACYSLLVTVAFIIFWV

SEQ ID NO: 53 - protein, 45 aa, Artificial Sequence
  source: /note=“Description of Artificial Sequence Synthetic polypeptide”
TTTPAPRPPTPAPTIASQPLSLRPEACRPAAGGAVHTRGLDFACD

SEQ ID NO: 54 - protein, 41 aa, Artificial Sequence
  source: /note=“Description of Artificial Sequence Synthetic polypeptide”
RSKRSRLLHSDYMNMTPRRPGPTRKHYQPYAPPRDFAAYRS

SEQ ID NO: 55 - protein, 112 aa, Artificial Sequence
  source: /note=“Description of Artificial Sequence Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 21 aa, Artificial Sequence
  source: /note=“Description of Artificial Sequence Synthetic peptide”
MALPVTALLLPLALLLHAARP

SEQ ID NO: 57 - DNA, 733 bp, Artificial Sequence
  source: /note=“Description of Artificial Sequence Synthetic polynucleotide”
caagtccagctgcagcagagcggcgccagcatgaagatgagctgtagagccagcggctacagcttcaccagctactggatccactggctgaagcagaggccagatcagggcctcgagtggatcggctatatcgatcctgccaccgcctacaccgagagcaaccagaagttcaaggacaaggccatcctgaccgccgacagaagcagcaacaccgccttcatgtacctgaacagcctgaccagcgaggacagcgccgtgtactattgcgccagagagagccccagactgcggagaggcatctactactacgccatggactattggggccagggcaccagcgtgacagtttcttctgcccctcaagccaagagcagcggcagcggatctgagtctaaggtggaccagagcgtggtcacccagcctccatctgctagcggaggacctggacagagcctgacaatcagctgtaccggcaccagctctgatgtcggcggctacaatcacgtgtcctggtatcagcagcaccccggcaaagcccctaagctgatgatctacgacgtgtccaagaggcctagcggcgtgccacacagattttccggcagcaagtctggcaataccgcctctctgaccgtgtctggactgcaggccgaagatgaggccgtgtattactgcagcagctacgccggctccaacaacctggtttttggcggaggcaccaaagtgaccgtgctgt

SEQ ID NO: 58 - DNA, 1477 bp, Artificial Sequence
  source: /note=“Description of Artificial Sequence Synthetic polynucleotide”
atggccttaccagtgaccgccttgctcctgccgctggccttgctgctccacgccgccaggccgggatctcaagtccagctgcagcagagcggcgccagcatgaagatgagctgtagagccagcggctacagcttcaccagctactggatccactggctgaagcagaggccagatcagggcctcgagtggatcggctatatcgatcctgccaccgcctacaccgagagcaaccagaagttcaaggacaaggccatcctgaccgccgacagaagcagcaacaccgccttcatgtacctgaacagcctgaccagcgaggacagcgccgtgtactattgcgccagagagagccccagactgcggagaggcatctactactacgccatggactattggggccagggcaccagcgtgacagtttcttctgcccctcaagccaagagcagcggcagcggatctgagagcaaggtggacgacatccagatgacccagacacctagcagcctgagcgcctctgtgggcgacagagtgaccatcacatgcagagccagccagagcatcagcagctttctgaactggtatcagcagaagcccggcaaggcccctaaactgctgatctacgccgccagcaatctgcagagcggagtgcctagcagattcagcggaagaggctccggcaccgatttcaccctgaccatatctagcctgcagccagaggacttcgccgcctactattgtcagcagggctacaccacacctctgacctttggccaggggaccaagctggaagtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 59 - DNA, 719 bp, Artificial Sequence
  source: /note=“Description of Artificial Sequence Synthetic polynucleotide”
caagtccagctgcagcagagcggcgccagcatgaagatgagctgtagagccagcggctacagcttcaccagctactggatccactggctgaagcagaggccagatcagggcctcgagtggatcggctatatcgatcctgccaccgcctacaccgagagcaaccagaagttcaaggacaaggccatcctgaccgccgacagaagcagcaacaccgccttcatgtacctgaacagcctgaccagcgaggacagcgccgtgtactattgcgccagagagagccccagactgcggagaggcatctactactacgccatggactattggggccagggcaccagcgtgacagtttcttctgcccctcaagccaagagcagcggcagcggatctgagagcaaggtggacgacatccagatgacccagacacctagcagcctgagcgcctctgtgggcgacagagtgaccatcacatgcagagccagccagagcatcagcagctttctgaactggtatcagcagaagcccggcaaggcccctaaactgctgatctacgccgccagcaatctgcagagcggagtgcctagcagattcagcggaagaggctccggcaccgatttcaccctgaccatatctagcctgcagccagaggacttcgccgcctactattgtcagcagggctacaccacacctctgacctttggccaggggaccaagctggaagt

SEQ ID NO: 60 - DNA, 1479 bp, Artificial Sequence
  source: /note=“Description of Artificial Sequence Synthetic polynucleotide”
atggccttaccagtgaccgccttgctcctgccgctggccttgctgctccacgccgccaggccgcaagtccagctgcagcagagcggcgccagcatgaagatgagctgtagagccagcggctacagcttcaccagctactggatccactggctgaagcagaggccagatcagggcctcgagtggatcggctatatcgatcctgccaccgcctacaccgagagcaaccagaagttcaaggacaaggccatcctgaccgccgacagaagcagcaacaccgccttcatgtacctgaacagcctgaccagcgaggacagcgccgtgtactattgcgccagagagagccccagactgcggagaggcatctactactacgccatggactattggggccagggcaccagcgtgacagtttcttctgcccctcaagccaagagcagcggcagcggatctgagagcaaagtggatagcagcgagctgacacaggaccccgctgtgtctgttgctctgggccagacagtgcggattacctgtcagggcgatagcctgcggagctactatgccagctggtatcagcagaagcccggacaggctcctgtgctggtcatctacggcaagaacaacaggcccagcggcatccccgatagattttctggcagcagctccggcaataccgccagcctgacaattactggcgcccaggccgaagatgaggccgactactactgcaacagcagagactccagcggcaatcacgtggtgtttggcggcggaacaaagctgacagtgctgtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 61 - protein, 496 aa, Artificial Sequence
  source: /note=“Description of Artificial Sequence Synthetic polypeptide”
MALPVTALLLPLALLLHAARPGSQVQLQQSGASMKMSCRASGYSFTSYWIHWLKQRPDQGLEWIGYIDPATAYTESNQKFKDKAILTADRSSNTAFMYLNSLTSEDSAVYYCARESPRLRRGIYYYAMDYWGQGTSVTVSSAPQAKSSGSGSESKVDQSVVTQPPSASGGPGQSLTISCTGTSSDVGGYNHVSWYQQHPGKAPKLMIYDVSKRPSGVPHRFSGSKSGNTASLTVSGLQAEDEAVYYCSSYAGSNNLVFGGGTKVTVLASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 491 aa, Artificial Sequence
  source: /note=“Description of Artificial Sequence Synthetic polypeptide”
MALPVTALLLPLALLLHAARPGSQVQLQQSGASMKMSCRASGYSFTSYWIHWLKQRPDQGLEWIGYIDPATAYTESNQKFKDKAILTADRSSNTAFMYLNSLTSEDSAVYYCARESPRLRRGIYYYAMDYWGQGTSVTVSSAPQAKSSGSGSESKVDDIQMTQTPSSLSASVGDRVTITCRASQSISSFLNWYQQKPGKAPKLLIYAASNLQSGVPSRFSGRGSGTDFTLTISSLQPEDFAAYYCQQGYTTPLTFGQGTKLEASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 494 aa, Artificial Sequence
  source: /note=“Description of Artificial Sequence Synthetic polypeptide”
MALPVTALLLPLALLLHAARPGSQVQLQQSGASMKMSCRASGYSFTSYWIHWLKQRPDQGLEWIGYIDPATAYTESNQKFKDKAILTADRSSNTAFMYLNSLTSEDSAVYYCARESPRLRRGIYYYAMDYWGQGTSVTVSSAPQAKSSGSGSESKVDSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - DNA, 1491 bp, Artificial Sequence
  source: /note=“Description of Artificial Sequence Synthetic polynucleotide”
atggccttaccagtgaccgccttgctcctgccgctggccttgctgctccacgccgccaggccgggatctcaagtccagctgcagcagagcggcgccagcatgaagatgagctgtagagccagcggctacagcttcaccagctactggatccactggctgaagcagaggccagatcagggcctcgagtggatcggctatatcgatcctgccaccgcctacaccgagagcaaccagaagttcaaggacaaggccatcctgaccgccgacagaagcagcaacaccgccttcatgtacctgaacagcctgaccagcgaggacagcgccgtgtactattgcgccagagagagccccagactgcggagaggcatctactactacgccatggactattggggccagggcaccagcgtgacagtttcttctgcccctcaagccaagagcagcggcagcggatctgagtctaaggtggaccagagcgtggtcacccagcctccatctgctagcggaggacctggacagagcctgacaatcagctgtaccggcaccagctctgatgtcggcggctacaatcacgtgtcctggtatcagcagcaccccggcaaagcccctaagctgatgatctacgacgtgtccaagaggcctagcggcgtgccacacagattttccggcagcaagtctggcaataccgcctctctgaccgtgtctggactgcaggccgaagatgaggccgtgtattactgcagcagctacgccggctccaacaacctggtttttggcggaggcaccaaagtgaccgtgctgtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 65 - protein, 18 aa, Artificial Sequence
  source: /note=“Description of Artificial Sequence Synthetic peptide”
EGRGSLLTCGDVEENPGP

SEQ ID NO: 66 - DNA, 727 bp, Artificial Sequence
  source: /note=“Description of Artificial Sequence Synthetic polynucleotide”
caagtccagctgcagcagagcggcgccagcatgaagatgagctgtagagccagcggctacagcttcaccagctactggatccactggctgaagcagaggccagatcagggcctcgagtggatcggctatatcgatcctgccaccgcctacaccgagagcaaccagaagttcaaggacaaggccatcctgaccgccgacagaagcagcaacaccgccttcatgtacctgaacagcctgaccagcgaggacagcgccgtgtactattgcgccagagagagccccagactgcggagaggcatctactactacgccatggactattggggccagggcaccagcgtgacagtttcttctgcccctcaagccaagagcagcggcagcggatctgagagcaaagtggatagcagcgagctgacacaggaccccgctgtgtctgttgctctgggccagacagtgcggattacctgtcagggcgatagcctgcggagctactatgccagctggtatcagcagaagcccggacaggctcctgtgctggtcatctacggcaagaacaacaggcccagcggcatccccgatagattttctggcagcagctccggcaataccgccagcctgacaattactggcgcccaggccgaagatgaggccgactactactgcaacagcagagactccagcggcaatcacgtggtgtttggcggcggaacaaagctgacagtgctgt

SEQ ID NO: 67 - protein, 244 aa, Artificial Sequence
  source: /note=“Description of Artificial Sequence Synthetic polypeptide”
QVQLQQSGASMKMSCRASGYSFTSYWIHWLKQRPDQGLEWIGYIDPATAYTESNQKFKDKAILTADRSSNTAFMYLNSLTSEDSAVYYCARESPRLRRGIYYYAMDYWGQGTSVTVSSAPQAKSSGSGSESKVDQSVVTQPPSASGGPGQSLTISCTGTSSDVGGYNHVSWYQQHPGKAPKLMIYDVSKRPSGVPHRFSGSKSGNTASLTVSGLQAEDEAVYYCSSYAGSNNLVFGGGTKVTVL

SEQ ID NO: 68 - protein, 239 aa, Artificial Sequence
  source: /note=“Description of Artificial Sequence Synthetic polypeptide”
QVQLQQSGASMKMSCRASGYSFTSYWIHWLKQRPDQGLEWIGYIDPATAYTESNQKFKDKAILTADRSSNTAFMYLNSLTSEDSAVYYCARESPRLRRGIYYYAMDYWGQGTSVTVSSAPQAKSSGSGSESKVDDIQMTQTPSSLSASVGDRVTITCRASQSISSFLNWYQQKPGKAPKLLIYAASNLQSGVPSRFSGRGSGTDFTLTISSLQPEDFAAYYCQQGYTTPLTFGQGTKLE

SEQ ID NO: 69 - protein, 242 aa, Artificial Sequence
  source: /note=“Description of Artificial Sequence Synthetic polypeptide”
QVQLQQSGASMKMSCRASGYSFTSYWIHWLKQRPDQGLEWIGYIDPATAYTESNQKFKDKAILTADRSSNTAFMYLNSLTSEDSAVYYCARESPRLRRGIYYYAMDYWGQGTSVTVSSAPQAKSSGSGSESKVDSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 70 - DNA, 63 bp, Artificial Sequence
  source: /note=“Description of Artificial Sequence Synthetic oligonucleotide”
atggccttaccagtgaccgccttgctcctgccgctggccttgctgctccacgccgccaggccg

SEQ ID NO: 71 - DNA, 57 bp, Artificial Sequence
  source: /note=“Description of Artificial Sequence Synthetic oligonucleotide”
atggactggatctggcgcatcctgtttctcgtgggagccgccacaggcgcccattct

SEQ ID NO: 72 - protein, 19 aa, Artificial Sequence
  source: /note=“Description of Artificial Sequence Synthetic peptide”
MDWIWRILFLVGAATGAHS

SEQ ID NO: 73 - protein, 25 aa, Artificial Sequence
  source: /note=“Description of Artificial Sequence Synthetic peptide”
MNRGVPFRHLLLVLQLALLPAATQG

SEQ ID NO: 74 - protein, 8 aa, Artificial Sequence
  source: /note=“Description of Artificial Sequence Synthetic peptide”
  MOD_RES (2)..(2): Any amino acid
  MOD_RES (4)..(4): Any amino acid
DXEXNPGP

SEQ ID NO: 75 - protein, 4 aa, Artificial Sequence
  source: /note=“Description of Artificial Sequence Synthetic peptide”
  source: /note=“See specification as filed for detailed description of substitutions and preferred embodiments”
GGGS

SEQ ID NO: 76 - protein, 20 aa, Artificial Sequence
  source: /note=“Description of Artificial Sequence Synthetic peptide”
GGGGSGGGGSGGGGSGGGGS

SEQ ID NO: 77 - protein, 15 aa, Artificial Sequence
  source: /note=“Description of Artificial Sequence Synthetic peptide”
GGGGSGGGGSGGGGS

SEQ ID NO: 78 - protein, 4 aa, Artificial Sequence
  source: /note=“Description of Artificial Sequence Synthetic peptide”
GGGS

SEQ ID NO: 79 - protein, 18 aa, Artificial Sequence
  source: /note=“Description of Artificial Sequence Synthetic peptide”
GSTSGSGKPGSGEGSTKG

SEQ ID NO: 80 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2023/0141511 A1
(43) Pub. Date: May 11, 2023

(54) CAR-T CELL THERAPY TARGETING NGCGM3

(71) Applicants: LUDWIG INSTITUTE FOR CANCER RESEARCH LTD, Zurich (CH); UNIVERSITY OF LAUSANNE, Lausanne (CH); CENTRE HOSPITALIER UNIVERSITAIRE VAUDOIS, Lausanne (CH)

(72) Inventors: George COUKOS, Épalinges (CH); Melita IRVING, Épalinges (CH); Greta Maria Paola Giordano ATTIANESE, Lausanne (CH)

(73) Assignees: LUDWIG INSTITUTE FOR CANCER RESEARCH LTD, Zurich (CH); UNIVERSITY OF LAUSANNE, Lausanne (CH); CENTRE HOSPITALIER UNIVERSITAIRE VAUDOIS, Lausanne (CH)

(21) Appl. No.: 17/787,384

(22) PCT Filed: Dec. 18, 2020
(86) PCT No.: PCT/IB2020/001051

§ 371 Date: Jun. 20, 2022

Related U.S. Application Data
(60) Provisional application No. 62/951,683, filed on Dec. 20, 2019.

(51) Int. Cl. A61K 35/17; C12N 15/86; C12N 5/0783; C07K 16/30; C07K 14/725; A61P 35/00

(52) U.S. Cl. CPC A61K 35/17; C12N 15/86; C12N 5/0636; C07K 16/30; C07K 14/7051; A61P 35/00; C12N 2740/15043; C07K 2317/24; C07K 2317/565; C07K 2319/03

(57) ABSTRACT

The invention relates to chimeric antigen receptors (CARs) targeting a cancer-associated antigen and their use for treatment of a tumor or cancer. In particular, the invention provides compositions and methods for treating diseases associated with the antigen NGcGM3. The invention also relates to CARs specific to NGcGM3, vectors encoding the same, and recombinant T cells comprising the CARs of the present invention. The invention also includes methods of administering a genetically modified T cell expressing a CAR that comprises an antigen binding domain that binds to NGcGM3.

### CROSS-REFERENCE TO RELATED APPLICATIONS

This application claims priority to U.S. Provisional Application No. 62/951,683, filed Dec. 20, 2019, the disclosure of which is herein incorporated by reference in its entirety.

### FIELD OF THE INVENTION

The invention relates to chimeric antigen receptors (CARs) targeting a cancer-associated antigen and their use for treatment. In particular, the invention provides compositions and methods for treating diseases associated with expression of a cancer-associated antigen as described herein. The invention also relates to CARs specific to a cancer-associated antigen as described herein, vectors encoding the same, and recombinant T cells comprising the CARs of the present invention. The invention also includes methods of administering a genetically modified T cell expressing a CAR that comprises an antigen binding domain that binds to a cancer-associated antigen as described herein.

### BACKGROUND

Chimeric antigen receptors (CARs) are hybrid molecules comprising an antigen-targeting moiety, typically a single-chain variable fragment (scFv), followed by a linker, transmembrane (TM) domain, and various endodomains (EDs) involved in T-cell activation. First generation CARs include the ED of CD3-zeta only, required for “signal 1” of T cell activation, while second and third generation CARs also have one or more co-stimulatory EDs, respectively, such as CD28 and 4-1BB, to provide “signal 2”.

The adoptive transfer of scFv-directed T lymphocytes, so-called CAR T cells, has emerged as a potent treatment against various advanced cancers. For example, recent clinical trials with CD19-targeted CAR T cells have yielded up to 90% complete remission rates for patients suffering advanced acute lymphoblastic leukemia (ALL), a “liquid” tumor [1-3]. “Solid” tumors, however, remain a significant challenge to CAR-based therapy. This is in part due to the fact that there are few bona fide tumor antigens that are not found on healthy tissue, and as such important “on-target/off-tumor” toxicities have occurred in CAR T cell treated patients, and in some instances even leading to death [4].

Thus, there remains a need for CAR-based therapies that specifically target tumor cells and do not cause harm to healthy tissue.

### SUMMARY OF THE INVENTION

In certain aspects, provided herein is an isolated nucleic acid molecule encoding a chimeric antigen receptor (CAR). The CAR comprises an anti-NGcGM3 binding domain, a transmembrane domain, and an endodomain.

In various embodiments, the encoded anti-NGcGM3 binding domain comprises an anti-NGcGM3 heavy chain variable domain sequence comprising: a heavy chain complementary determining region 1 (HC CDR1) sequence SYWIH (SEQ ID NO:3), a heavy chain complementary determining region 2 (HC CDR2) sequence YIDPATAYTESNQKFKD (SEQ ID NO:5), and a heavy chain complementary determining region 3 (HC CDR3) sequence ESPRLRRGIYYYAMDY (SEQ ID NO:7).

In some embodiments, the encoded anti-NGcGM3 heavy chain variable domain sequence comprises: a heavy chain complementary determining region 1 (HC CDR1) sequence SYWIH (SEQ ID NO:3), a heavy chain complementary determining region 2 (HC CDR2) sequence YIDPATAYTESNQKFKD (SEQ ID NO:5), heavy chain complementary determining region 3 (HC CDR3) sequence ESPRLRRGIYYYAMDY (SEQ ID NO:7), a framework region 1 (FR1) sequence QVQLQQSGASMKMSCRASGYSFT (SEQ ID NO:2), a framework region 2 (FR2) sequence WLKQRPDQGLEWIG (SEQ ID NO:4), a framework region 3 (FR3) sequence KAILTADRSSNTAFMYLNSLTSEDSAVYYCAR (SEQ ID NO:6), and a framework region 4 (FR4) sequence WGQGTSVTVSS (SEQ ID NO:8).

In various embodiments, the encoded anti-NGcGM3 binding domain comprises an anti-NGcGM3 heavy chain variable domain amino acid sequence of SEQ ID NO:1, or a sequence with at least 80% identity thereof.

In some embodiments, the encoded NGcGM3 binding domain comprises an anti-NGcGM3 light chain variable domain sequence comprising a light chain complementarity determining region 1 (LC CDR1) amino acid sequence TGTSSDVGGYNHVS (SEQ ID NO:18), RASQSISSFLN (SEQ ID NO:25), or QGDSLRSYYAS (SEQ ID NO:32). In various embodiments, the NGcGM3 binding domain comprises an anti-NGcGM3 light chain variable domain sequence comprising a light chain complementarity determining region 2 (LC CDR2) amino acid sequence DVSKRPS (SEQ ID NO:20), AASNLQS (SEQ ID NO:27), or GKNNRPS (SEQ ID NO:34). In some embodiments, the NGcGM3 binding domain comprises an anti-NGcGM3 light chain variable domain sequence comprising a light chain complementarity determining region 3 (LC CDR3) amino acid sequence SSYAGSNNLVF (SEQ ID NO:22), QQGYTTPLTF (SEQ ID NO:29), or NSRDSSGNHVVF (SEQ ID NO:36).

In various embodiments, the encoded anti-NGcGM3 binding domain comprises an anti-NGcGM3 light chain variable domain amino acid sequence comprising: a light chain complementary determining region 1 (LC CDR1) sequence TGTSSDVGGYNHVS (SEQ ID NO:18), a light chain complementary determining region 2 (LC CDR2) sequence DVSKRPS (SEQ ID NO:20), and a light chain complementary determining region 3 (LC CDR3) sequence SSYAGSNNLVF (SEQ ID NO:22), a light chain complementary determining region 1 (LC CDR1) sequence RASQSISSFLN (SEQ ID NO:25), a light chain complementary determining region 2 (LC CDR2) sequence AASNLQS (SEQ ID NO:27), and a light chain complementary determining region 3 (LC CDR3) sequence QQGYTTPLTF (SEQ ID NO:29), or a light chain complementary determining region 1 (LC CDR1) sequence QGDSLRSYYAS (SEQ ID NO:32), a light chain complementary determining region 2 (LC CDR2) sequence GKNNRPS (SEQ ID NO:34), and a light chain complementary determining region 3 (LC CDR3) sequence NSRDSSGNHVVF (SEQ ID NO:36).

In some embodiments, the encoded anti-NGcGM3 light chain variable domain amino acid sequence comprises: (i) a light chain complementary determining region 1 (LC CDR1) sequence TGTSSDVGGYNHVS (SEQ ID NO:18), a light chain complementary determining region 2 (LC CDR2) sequence DVSKRPS (SEQ ID NO:20), a light chain complementary determining region 3 (LC CDR3) sequence SSYAGSNNLVF (SEQ ID NO:22), a framework region 1 (FR1) sequence QSVVTQPPSASGGPGQSLTISC (SEQ ID NO:17), a framework region 2 (FR2) sequence WYQQHPGKAPKLMIY (SEQ ID NO:19), a framework region 3 (FR3) sequence GVPHRFSGSKSGNTASLTVSGLQAEDEAVYYC (SEQ ID NO:21), and a framework region 4 (FR4) sequence GGGTKVTVL (SEQ ID NO:23); or (ii) a light chain complementary determining region 1 (LC CDR1) sequence RASQSISSFLN (SEQ ID NO:25), a light chain complementary determining region 2 (LC CDR2) sequence AASNLQS (SEQ ID NO:27), a light chain complementary determining region 3 (LC CDR3) sequence QQGYTTPLTF (SEQ ID NO:29), a framework region 1 (FR1) sequence DIQMTQTPSSLSASVGDRVTITC (SEQ ID NO:24), a framework region 2 (FR2) sequence WYQQKPGKAPKLLIY (SEQ ID NO:26), a framework region 3 (FR3) sequence GVPSRFSGRGSGTDFTLTISSLQPEDFAAYYC (SEQ ID NO:28), and a framework region 4 (FR4) sequence GQGTKLE (SEQ ID NO:30); or (iii) a light chain complementary determining region 1 (LC CDR1) sequence QGDSLRSYYAS (SEQ ID NO:32), a light chain complementary determining region 2 (LC CDR2) sequence GKNNRPS (SEQ ID NO:34), a light chain complementary determining region 3 (LC CDR3) sequence NSRDSSGNHVVF (SEQ ID NO:36), a framework region 1 (FR1) sequence SSELTQDPAVSVALGQTVRITC (SEQ ID NO:31), a framework region 2 (FR2) sequence WYQQKPGQAPVLVIY (SEQ ID NO:33), a framework region 3 (FR3) sequence GIPDRFSGSSSGNTASLTITGAQAEDEADYYC (SEQ ID NO:35), and a framework region 4 (FR4) sequence GGGTKLTVL (SEQ ID NO:37).

In some embodiments, the encoded anti-NGcGM3 binding domain comprises an anti-NGcGM3 light chain variable domain amino acid sequence comprising SEQ ID NO:9, or a sequence with at least 80% identity thereof.

In various embodiments, the encoded anti-NGcGM3 binding domain comprises an anti-NGcGM3 light chain variable domain amino acid sequence of SEQ ID NO:10, or a sequence with at least 80% identity thereof.

In some embodiments, the encoded anti-NGcGM3 binding domain comprises an anti-NGcGM3 light chain variable domain amino acid sequence of SEQ ID NO:11, or a sequence with at least 80% identity thereof.

In various embodiments, isolated nucleic acid molecule comprises a nucleotide sequence encoding said anti-NGcGM3 binding domain comprising SEQ ID NO:57, or a sequence with at least 80% identity thereof.

In some embodiments, the encoded anti-NGcGM3 binding domain comprises a linker between the heavy chain variable domain and the light chain variable domain. In various embodiments, the linker comprises an amino acid sequence APQAKSSGSGSESKVD (SEQ ID NO:16), or a sequence with at least 80% identity thereof.

In some embodiments, the isolated nucleic acid molecule comprises a nucleotide sequence encoding the anti-NGcGM3 binding domain comprising SEQ ID NO:59, SEQ ID NO:60, or SEQ ID NO:61, or a sequence with at least 80% identity to SEQ ID NO:59, SEQ ID NO:60, or SEQ ID NO:61.

In some embodiments, the isolated nucleic acid molecule comprises a nucleotide sequence encoding the anti-NGcGM3 binding domain comprising SEQ ID NO:59, or a sequence with at least 80% identity thereof.

In various embodiments, the isolated nucleic acid molecule comprises a nucleotide sequence encoding the anti-NGcGM3 binding domain comprising SEQ ID NO:66, or a sequence with at least 80% identity thereof.

In various embodiments, the encoded anti-NGcGM3 binding domain comprises an amino acid sequence of SEQ ID NO:67, SEQ ID NO:68, SEQ ID NO:69, or a sequence with at least 80% identity to SEQ ID NO:67, SEQ ID NO:68, or SEQ ID NO:69.

In various embodiments, the encoded anti-NGcGM3 binding domain comprises an amino acid sequence of SEQ ID NO:67, or a sequence with at least 80% identity to SEQ ID NO:67.

In various embodiments, the encoded anti-NGcGM3 binding domain comprises an amino acid sequence of SEQ ID NO:68, or a sequence with at least 80% identity to SEQ ID NO:68.

In various embodiments, the encoded anti-NGcGM3 binding domain comprises an amino acid sequence of SEQ ID NO:69, or a sequence with at least 80% identity to SEQ ID NO:69.

In some embodiments, the encoded transmembrane domain comprises a transmembrane domain of the alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137 or CD154. In various embodiments, the encoded transmembrane domain comprises an amino acid sequence of SEQ ID NO:52, or a sequence with at least 80% identity thereof. In some embodiments, a nucleotide sequence encoding the transmembrane domain comprises SEQ ID NO:49, or a sequence with at least 80% identity thereof.

In various embodiments, the encoded anti-NGcGM3 binding domain is operably linked to the transmembrane domain via a hinge domain. In some embodiments, the hinge domain comprises an amino acid sequence of SEQ ID NO:53, or a sequence with at least 80% identity thereof. In various embodiments, a nucleotide sequence encoding the hinge domain comprises SEQ ID NO:51, or a sequence with at least 80% identity thereof.

In some embodiments, the encoded endodomain comprises an intracellular (IC) domain comprising a sequence derived from DAP10, DAP12, Fc epsilon receptor I gamma chain (FCER1G), FcR beta CD3-delta, CD3-epsilon, CD3-gamma, CD3-zeta, CD226, CD66d, CD79A, or CD79B. In various embodiments, the IC domain comprises an amino acid sequence of SEQ ID NO:54, or a sequence with at least 80% identity thereof. In some embodiments, a nucleotide sequence encoding the IC domain comprises SEQ ID NO:48, or a sequence with at least 80% identity thereof.

In various embodiments, the encoded endodomain comprises a signalling domain derived from DAP10, DAP12, Fc epsilon receptor I gamma chain (FCER1G), FcR beta CD3-delta, CD3-epsilon, CD3-gamma, CD3-zeta, CD226, CD66d, CD79A, or CD79B. In some embodiments, the encoded signalling domain comprises an amino acid sequence of SEQ ID NO:55, or a sequence with at least 80% identity thereof. In various embodiments, a nucleotide sequence encoding the signalling domain comprises SEQ ID NO:50, or a sequence with at least 80% identity thereof.

In various embodiments, the encoded CAR further comprises a leader sequence. In some embodiments, the leader sequence comprises an amino acid sequence of SEQ ID NO:56, or a sequence with at least 80% identity thereof. In various embodiments, a nucleotide sequence encoding the leader sequence comprises SEQ ID NO:70, or a sequence with at least 80% identity thereof.

In various embodiments, the nucleic acid molecule is a DNA molecule. In some embodiments, the nucleic acid molecule is an RNA molecule.

In certain aspects, provided herein is an isolated polypeptide molecule encoded by the nucleic acid molecule.

In certain aspects, provided herein is an isolated chimeric antigen receptor (CAR) molecule, wherein said CAR comprises an anti-NGcGM3 binding domain, a transmembrane domain, and an endodomain.

In various embodiments, the NGcGM3 binding domain of the isolated CAR molecule comprises an anti-NGcGM3 heavy chain variable domain sequence comprising: a heavy chain complementary determining region 1 (HC CDR1) sequence SYWIH (SEQ ID NO:3), a heavy chain complementary determining region 2 (HC CDR2) sequence YIDPATAYTESNQKFKD (SEQ ID NO:5), and a heavy chain complementary determining region 3 (HC CDR3) sequence ESPRLRRGIYYYAMDY (SEQ ID NO:7). In some embodiments, the anti-NGcGM3 binding domain comprises an anti-NGcGM3 heavy chain variable domain sequence comprising: a heavy chain complementary determining region 1 (HC CDR1) sequence SYWIH (SEQ ID NO:3), a heavy chain complementary determining region 2 (HC CDR2) sequence YIDPATAYTESNQKFKD (SEQ ID NO:5), a heavy chain complementary determining region 3 (HC CDR3) sequence ESPRLRRGIYYYAMDY (SEQ ID NO:7), a framework region 1 (FR1) sequence QVQLQQSGASMKMSCRASGYSFT (SEQ ID NO:2), a framework region 2 (FR2) sequence WLKQRPDQGLEWIG (SEQ ID NO:4), a framework region 3 (FR3) sequence KAILTADRSSNTAFMYLNSLTSEDSAVYYCAR (SEQ ID NO:6) and a framework region 4 (FR4) sequence WGQGTSVTVSS (SEQ ID NO:8).

In some embodiments, the anti-NGcGM3 binding domain of the isolated CAR molecule comprises an anti-NGcGM3 heavy chain variable domain amino acid sequence of SEQ ID NO:1.

In various embodiments, the NGcGM3 binding domain of the isolated CAR molecule comprises an anti-NGcGM3 light chain variable domain sequence comprising a light chain complementarity determining region 1 (LC CDR1) sequence TGTSSDVGGYNHVS (SEQ ID NO:18), RASQSISSFLN (SEQ ID NO:25), or QGDSLRSYYAS (SEQ ID NO:32). In some embodiments, the NGcGM3 binding domain of the isolated CAR molecule comprises an anti-NGcGM3 light chain variable domain sequence comprising a light chain complementarity determining region 2 (LC CDR2) sequence DVSKRPS (SEQ ID NO:20), AASNLQS (SEQ ID NO:27), or GKNNRPS (SEQ ID NO:34). In various embodiments, the NGcGM3 binding domain of the isolated CAR molecule comprises an anti-NGcGM3 light chain variable domain sequence comprising a light chain complementarity determining region 3 (LC CDR3) sequence SSYAGSNNLVF (SEQ ID NO:22), QQGYTTPLTF (SEQ ID NO:29), or NSRDSSGNHVVF (SEQ ID NO:36).

In various embodiments, the anti-NGcGM3 binding domain of the isolated CAR molecule comprises an anti-NGcGM3 light chain variable domain sequence comprising: (i) a light chain complementary determining region 1 (LC CDR1) sequence TGTSSDVGGYNHVS (SEQ ID NO:18), a light chain complementary determining region 2 (LC CDR2) sequence DVSKRPS (SEQ ID NO:20), and a light chain complementary determining region 3 (LC CDR3) sequence SSYAGSNNLVF (SEQ ID NO:22); or (ii) a light chain complementary determining region 1 (LC CDR1) sequence RASQSISSFLN (SEQ ID NO:25), a light chain complementary determining region 2 (LC CDR2) sequence AASNLQS (SEQ ID NO:27), and a light chain complementary determining region 3 (LC CDR3) sequence QQGYTTPLTF (SEQ ID NO:29), or (iii) a light chain complementary determining region 1 (LC CDR1) sequence QGDSLRSYYAS (SEQ ID NO:32), a light chain complementary determining region 2 (LC CDR2) sequence GKNNRPS (SEQ ID NO:34), and a light chain complementary determining region 3 (LC CDR3) sequence NSRDSSGNHVVF (SEQ ID NO:36).

In some embodiments, the anti-NGcGM3 binding domain of the isolated CAR molecule comprises an anti-NGcGM3 light chain variable domain sequence comprising: (i) a light chain complementary determining region 1 (LC CDR1) sequence TGTSSDVGGYNHVS (SEQ ID NO:18), a light chain complementary determining region 2 (LC CDR2) sequence DVSKRPS (SEQ ID NO:20), a light chain complementary determining region 3 (LC CDR3) sequence SSYAGSNNLVF (SEQ ID NO:22), a framework region 1 (FR1) sequence QSVVTQPPSASGGPGQSLTISC (SEQ ID NO:17), a framework region 2 (FR2) sequence WYQQHPGKAPKLMIY (SEQ ID NO:19), a framework region 3 (FR3) sequence GVPHRFSGSKSGNTASLTVSGLQAEDEAVYYC (SEQ ID NO:21) and a framework region 4 (FR4) sequence GGGTKVTVL (SEQ ID NO:23); or (ii) a light chain complementary determining region 1 (LC CDR1) sequence RASQSISSFLN (SEQ ID NO:25), a light chain complementary determining region 2 (LC CDR2) sequence AASNLQS (SEQ ID NO:27), a light chain complementary determining region 3 (LC CDR3) sequence QQGYTTPLTF (SEQ ID NO:29), a framework region 1 (FR1) sequence DIQMTQTPSSLSASVGDRVTITC (SEQ ID NO:24), a framework region 2 (FR2) sequence WYQQKPGKAPKLLIY (SEQ ID NO:26), a framework region 3 (FR3) sequence GVPSRFSGRGSGTDFTLTISSLQPEDFAAYYC (SEQ ID NO:28) and a framework region 4 (FR4) sequence GQGTKLE (SEQ ID NO:30); or (iii) a light chain complementary determining region 1 (LC CDR1) sequence QGDSLRSYYAS (SEQ ID NO:32), a light chain complementary determining region 2 (LC CDR2) sequence GKNNRPS (SEQ ID NO:34), a light chain complementary determining region 3 (LC CDR3) sequence NSRDSSGNHVVF (SEQ ID NO:36), a framework region 1 (FR1) sequence SSELTQDPAVSVALGQTVRITC (SEQ ID NO:31), a framework region 2 (FR2) sequence WYQQKPGQAPVLVIY (SEQ ID NO:33), a framework region 3 (FR3) sequence GIPDRFSGSSSGNTASLTITGAQAEDEADYYC (SEQ ID NO:35) and a framework region 4 (FR4) sequence GGGTKLTVL (SEQ ID NO:37).

In some embodiments, the anti-NGcGM3 binding domain of the isolated CAR molecule comprises an anti-NGcGM3 light chain variable domain amino acid sequence of SEQ ID NO:9, or a sequence with at least 80% identity thereof.

In various embodiments, the anti-NGcGM3 binding domain of the isolated CAR molecule comprises an anti-NGcGM3 light chain variable domain amino acid sequence of SEQ ID NO:10, or a sequence with at least 80% identity thereof.

In some embodiments, the anti-NGcGM3 binding domain of the isolated CAR molecule comprises an anti-NGcGM3 light chain variable domain amino acid sequence of SEQ ID NO:11, or a sequence with at least 80% identity thereof.

In various embodiments, a nucleotide sequence encoding the anti-NGcGM3 binding domain of the isolated CAR molecule comprises SEQ ID NO:57, or a sequence with at least 80% identity thereof.

In some embodiments, a nucleotide sequence encoding said anti-NGcGM3 binding domain of the isolated CAR molecule comprises SEQ ID NO:59, or a sequence with at least 80% identity thereof.

In various embodiments, a nucleotide sequence encoding said anti-NGcGM3 binding domain of the isolated CAR molecule comprises SEQ ID NO:66, or a sequence with at least 80% identity thereof.

In some embodiments, the anti-NGcGM3 binding domain of the isolated CAR molecule comprises a linker between the heavy chain variable domain and the light chain variable domain. In various embodiments, the linker comprises an amino acid sequence APQAKSSGSGSESKVD (SEQ ID NO:16), or a sequence with at least 80% identity thereof.

In some embodiments, anti-NGcGM3 binding domain of the isolated CAR molecule comprises an amino acid sequence of SEQ ID NO:67, SEQ ID NO:68, SEQ ID NO:69, or a sequence with at least 80% identity to SEQ ID NO:67, SEQ ID NO:68, or SEQ ID NO:69.

In various embodiments, the isolated CAR molecule includes a transmembrane domain derived from the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 or CD154. In some embodiments, the transmembrane domain comprises an amino acid sequence of SEQ ID NO:52, or a sequence with at least 80% identity thereof. In various embodiments, a nucleotide sequence encoding the transmembrane domain comprises a sequence of SEQ ID NO:49, or a sequence with at least 80% identity thereof.

In some embodiments, the anti-NGcGM3 binding domain of the isolated CAR molecule is operably linked to the transmembrane domain via a hinge domain. In various embodiments, the hinge domain comprises an amino acid sequence of SEQ ID NO:53, or a sequence with at least 80% identity thereof. In some embodiments, a nucleotide sequence encoding the hinge domain comprises SEQ ID NO:51, or a sequence with at least 80% identity thereof.

In various embodiments, the endodomain of the isolated CAR molecule comprises an intracellular (IC) domain comprising a sequence derived from DAP10, DAP12, Fc epsilon receptor I gamma chain (FCER1G), FcR beta CD3-delta, CD3-epsilon, CD3-gamma, CD3-zeta, CD226, CD66d, CD79A, or CD79B. In some embodiments, the IC domain comprises an amino acid sequence of SEQ ID NO:54, or a sequence with at least 80% identity thereof. In various embodiments, a nucleotide sequence encoding the IC domain comprises SEQ ID NO:48, or a sequence with at least 80% identity thereof.

In some embodiments, the endodomain of the isolated CAR molecule comprises a signalling domain that comprises a sequence derived from DAP10, DAP12, Fc epsilon receptor I gamma chain (FCER1G), FcR beta CD3-delta, CD3-epsilon, CD3-gamma, CD3-zeta, CD226, CD66d, CD79A, or CD79B. In some embodiments, the signalling domain comprises an amino acid sequence of SEQ ID NO:55, or a sequence with at least 80% identity thereof. In various embodiments, a nucleotide sequence encoding the signalling domain comprises SEQ ID NO:50, or a sequence with at least 80% identity thereof.

In various embodiments, the isolated CAR molecule comprises a leader sequence. In some embodiments, the leader sequence comprises an amino acid sequence of SEQ ID NO:56, or a sequence with at least 80% identity thereof. In some embodiments, a nucleotide sequence encoding the leader sequence comprises SEQ ID NO:70, or a sequence with at least 80% identity thereof.

In certain aspects, provided herein is a vector comprising a nucleic acid molecule encoding the isolated CAR molecule.

In various embodiments, the vector is a DNA, an RNA, a plasmid, a lentivirus vector, an adenoviral vector, or a retrovirus vector. In some embodiments, the vector is a viral vector. In various embodiments, the vector is a lentivirus vector.

In some embodiments, the vector comprises a promoter. In various embodiments, the promoter is a T lymphocyte-specific promoter or an NK cell-specific promoter. In some embodiments, the promoter is a PGK promoter. In various embodiments, the PGK promoter comprises a nucleotide sequence of SEQ ID NO:47, or a sequence with at least 80% identity thereto.

In some embodiments, the vector is an in vitro transcribed vector.

In certain aspects, provided herein is a cell comprising the vector.

In some embodiments, the cell is a T cell. In various embodiments, the T cell is a CD8+ T cell. In some embodiments, the cell is a human cell.

In certain aspects, provided herein is a method of making a cell comprising transducing a T cell with the vector.

In certain aspects, provided herein is a method of providing an anti-tumor immunity in a mammal comprising administering to the mammal an effective amount of a cell expressing the isolated CAR molecule.

In various embodiments, the cell of one of the above methods is an autologous T cell. In some embodiments, the cell is an allogeneic T cell. In various embodiments, the mammal is a human.

In certain aspects, provided herein is a method of treating a mammal having a disease associated with expression of NGcGM3 comprising administering to the mammal an effective amount of cells expressing the isolated CAR molecule.

In various aspects, the disease associated with the expression of NGcGM3 is solid malignancies, carcinomas, lymphomas, sarcomas, blastomas, leukemias, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies, EBV-associated Hodgkin's, non-Hodgkin's lymphoma, post-transplant lymphomas, post-transplant lymphoproliferative disorder (PTLD), uterine cancer, renal cell carcinoma, hepatoma, hepatoblastoma, cancers of the bladder, blood, bone, bone marrow, brain, breast, colon, esophagus, gastrointestine, gum, head, kidney, liver, lung, nasopharynx, neck, ovary, prostate, skin, stomach, testis, tongue, uterus or any combination thereof. In some embodiments, the disease associated with NGcGM3 is a solid tumor. In various aspects, the solid tumor is an ovarian tumor.

In certain aspects, provided herein is the isolated nucleic acid molecule, the isolated CAR molecule, the vector, or any of the above cells for use as a medicament.

In certain aspects, provided herein is the isolated nucleic acid molecule, the isolated CAR molecule, the vector, or any of the above cells for use in the treatment of a disease expressing NGcGM3.

In certain aspects, provided herein is an isolated CAR molecule comprising an amino acid sequence of SEQ ID NO:61, or a sequence with at least 80% identity thereof.

In certain aspects, provided herein is an isolated CAR molecule comprising an amino acid sequence of SEQ ID NO:62, or a sequence with at least 80% identity thereof.

In certain aspects, provided herein is an isolated CAR molecule comprising an amino acid sequence of SEQ ID NO:63, or a sequence with at least 80% identity thereof.

In certain aspects, provided herein is a nucleic acid sequence encoding a CAR molecule comprising a nucleic acid sequence of SEQ ID NO:64, or a sequence with at least 80% identity thereof.

In certain aspects, provided herein is a nucleic acid sequence encoding a CAR molecule comprising a nucleic acid sequence of SEQ ID NO:58, or a sequence with at least 80% identity thereof.

In certain aspects, provided herein is a nucleic acid sequence encoding a CAR molecule comprising a nucleic acid sequence of SEQ ID NO:60, or a sequence with at least 80% identity thereof.

These and other aspects of the present invention will be apparent to those of ordinary skill in the art in the following description, claims and drawings.

### BRIEF DESCRIPTION OF THE DRAWINGS

FIGS. 1A-1G demonstrate that humanized anti-NGcGM3 CAR expressing T cells (primary and Jurkat T cells) recognize a NGcGM3 positive human tumor cell line in vitro. FIG. 1A is a schematic map of a lentiviral vector encoding humanized 14F7-derived CARs. Three variants of 14F7-derived humanized CARs (SEQ ID NO:61-63) were prepared: heavy chain (SEQ ID NO:1) and one of three 3 humanized variants of light chain murine 14F7 mAb [7Ah (SEQ ID NO:9), 7Bh (SEQ ID NO:10) and 8Bh (SEQ ID NO:11)] were cloned in-frame into a lentiviral vector encoding CD8 leader sequence (SEQ ID NO:56), a hinge domain (SEQ ID NO:53), a CD28 TM domain (SEQ ID NO:52), a CD28 intracellular (IC) domain (SEQ ID NO 54), and a CD3-zeta signalling domain (SEQ ID NO:55) to provide 7Ah (SEQ ID NO:61), 7Bh (SEQ ID NO:62), and 8Bh (SEQ ID NO:63) CARs, each under the control of a PGK promoter (SEQ ID NO:47). A gene reporter GFP was also cloned into the lentiviral vector in a T2A-mediated bicistronic manner. FIG. 1B is a flow cytometry cytogram demonstrating that Jurkat cells engineered with marker m-Cherry under the control of NFAT promoter (Jurkat-NFAT-mCherry) physiologically express NGcGM3. Isotype control is shown in grey. FIG. 1C is a compilation of flow cytometry cytograms collectively demonstrating that Jurkat-NFAT-mCherry cells transduced with the 3 variants of 14F7-derived humanized CARs (SEQ ID NO:61-63), thereby rendering the Jurkat-NFAT-mCherry cells CAR T cells, undergo reciprocal antigen-induced activation. Jurkat-NFAT-mCherry were transduced with the 3 variants of 14F7-derived humanized CARs (SEQ ID NO:61-63) and cultured with the addition or not of PMA/Iono (positive control of Jurkat-NFAT-mCherry activation) and analyzed after 48h for GFP and mCherry expression. FIG. 1D is a bar graph plotting the percent cells transduced with and expressing (as measured by GFP reporter expression) each 14F7-derived humanized CAR variant (SEQ ID NO:61-63). Primary human T cells were transduced with the 3 variants of humanized 14F7 derived CARs and the expression of the reporter gene GFP was assessed by flow cytometry. FIG. 1E is a flow cytometry cytogram demonstrating expression of NGcGM3 on human ovarian tumor cell line SKOV3 wild-type (SKOV3 wt.). Isotype control is in grey. FIG. 1F is a bar providing a quantification of humanized 14F7 derived CAR T cell IFN-gamma production upon 24h co-culture with SKOV3 wt. (p=0.0035 comparing 7Bh with untransduced cells (UTD), student t test). FIG. 1G is a scatter plot showing tumor killing assessment by dead cell count/mm² with IncuCyte instrument (**p=0.0022 comparing 8Bh CAR T cells with 7Ah and 7Bh, Mann-Whitney u test) (vertical bars represent average+/−SD).

FIGS. 2A-2H demonstrate that humanized NGcGM3-specific CAR T cells control NGcGM3-positive SKOV3 CMAH tumor growth in vivo. The left panel of FIG. 2A is a box plot demonstrating an increase in expression levels (*p=0.0188, Mann-Whitney u test) of NGcGM3 on subcutaneous tumors derived from human ovarian tumor cell line SKOV3 transduced to overexpress the enzyme CMAH (SKOV3 CMAH) relative to wt. cells (SKOV3). The middle and right panels of FIG. 2A are representative immunohistochemical images of the SKOV3 CMAH and SKOV3 cell lines (DAPI staining for nuclei and Allophycocyanin (APC) labeled secondary Ab specific for 14F7 anti-NGcGM3 Ab). FIG. 2B is a schematic illustrating an experimental design for an experiment wherein NSG female mice (n=6 animals/group) were subcutaneously injected with 5×10⁶ SKOV3 CMAH and subsequently treated after 4 days with peritumoral injections of 3×10⁶ CAR T or with peritumoral injections of UTD cells suspended in saline as a control. FIG. 2C is a plot of tumor growth measured by volume calipering over time (**** indicates p<0.0001, Two-way ANOVA with multiple comparison) (bars represent average+/−sem). FIG. 2D is a bar graph plotting tumor volumes at 15 days (** indicates p<0.01, Two-way ANOVA with multiple comparison). FIG. 2E is a bar graph summarizing results from ex vivo analysis of collected SKOV3 CMAH tumors for tumor weight. FIG. 2F is a box plot summarizing detection of total human CD3⁺ (normalized for tumor volume) within tumors, as determined using anti-CD3 Ab, as a percent of total live cells. FIG. 2G is a bar graph plotting GFP⁺ cells (right panel) within tumors as a percentage of total CD3⁺ cells (p<0.01, Mann-Whitney u test) (shown is average+/−SD). FIG. 2H is a plot demonstrating a correlation between tumor weight and human CD3⁺ T cell infiltration (r=−0.7778, p<0.0001, Pearson correlation)

### DETAILED DESCRIPTION

The present invention is based on the development of a CAR T cell therapy targeting a NGcGM3 ganglioside antigen that effectively and selectively kills cancerous cells.

All cells have surfaces coated by glycans (glycoproteins and glycolipids) that mediate biological processes, such as cell-cell interactions and recognition of extracellular molecules [5]. Tumor cells have a different glycosylation pattern than normal cells and this contributes to tumor growth, progression and immune-suppression. This is referred to as the so-called “sweet escape” [6]. Cancer associated glycans, in particular sialic acids, are “self-associated molecular patterns” (SAMPs) that inhibit innate immune cell activation and function by binding sialic acid-binding immunoglobulin-like lectins (SIGLECs) [7].

Gangliosides, or glycosphingolipids, comprise a ceramide linked to an oligosaccharide, with at least one residue of sialic acid (monosaccharide with a C9 backbone) [8]. The hydrophobic ceramide component of gangliosides is anchored to the cellular membrane, while the hydrophilic glycan component is presented at the cell surface. One of the most common sialic acids is N-acetylneuraminic acid (NAc) and it is found on surfaces of almost all healthy tissue cells. The N-acetyl group can be hydroxylated by the enzyme cytidine monophospho-N-acetyl-neuraminic acid hydroxylase (CMP-NAc hydroxylase; CMAH) generating N-glycolylneuraminic acid (NGc). NGc sialo-conjugates are not detected on the cell surface of healthy human cells, because the human genome (as well the genomes of chickens and new-world primates) lacks a functional CMAH, due to a 92 base pair deletion in exon 6 of a CMAH coding region sequence [9]. Conversely, NGc-gangliosides, in particular ganglioside monosialic 3 [39] (NGcGM3; GM3(Neu5Gc) ganglioside; C₆₅H₁₂₁N₃O₂₂; CAS Number 2260670-78-6) is exposed at the cell surface of several types of solid tumors, including breast [10,11], epithelial digestive [12] and genitourinary [13] tracts cancers, retinoblastoma [14], non-small cells lung cancer (NSCLC) [14], and melanoma [15], and it is currently used as poor-prognosis marker. The chemical formula (chemical formula I) of NGcGM3 is:

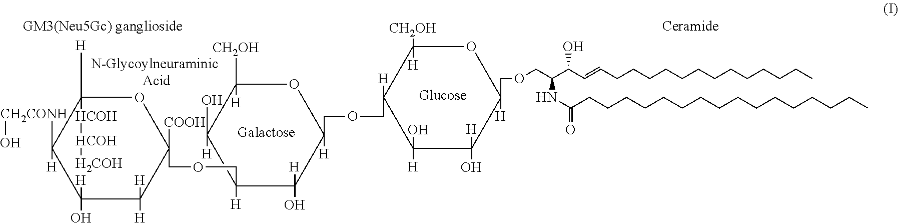

NGcGM3 has been detected in fetal samples—as it was originally classified as an oncofetal antigen.

Given that there is a CMAH deletion in the human genome, it was proposed that NGc can be produced by an alternate pathway that does not require CMAH. With a Cmah−/− mouse model, it was demonstrated that NGc present on human tumor cells (and fetus) most likely derives from dietary sources, since NGc can be assimilated by micropinocytosis [16]. NGc comes from mammalian derived food (particularly red meat or dairy products) and can be incorporated into a cell surface, therefore expression of this sialic acid on tumor cells has been attributed to their high rate of metabolism. Moreover, hypoxia induces upregulation of the gene for sialin, or soluble carrier 17A5 (SLC17A5), a transporter for sialic acid, thus tumor related hypoxia contributes to NGc expression on tumor cells' surface [17,18].

NGcGM3 is immunogenic. In adults (both healthy or cancer patients) 0.1-0.2% of total serum antibodies are IgA, IgM or IgGs specific for this sialic acid. While dietary NGc alone is likely not enough to generate these antibodies, xeno-anitbodies against NGc are induced in the first year of life by resident commensal bacteria that present the sialic acid at their cell surface upon incorporation from the diet [19]. Also, NGcGM3 has an immune-suppressive effect including downregulating the expression of CD4 in T cells, decreasing T cell cytokines production, and decreasing T cell proliferative capacity. In addition, NGcGM3 impairs dendric cell maturation [20].

NGcGM3 is, therefore, an appealing target for immunotherapy. Several clinical trials with racotumomab (an anti-idiotype monoclonal antibody that was obtained by immunizing mice with an IgM specific for NGc-containing gangliosides [21]) administered as a vaccine (precipitated in aluminum hydroxide) in melanoma [22], breast [23] and NSCLC [24,25] cancer patients showed that racotumomab caused an increase in overall survival. Vaccination with racotumomab activates an anti-idiotype response in the patients, which is possible because anti-NGcGM3 antibodies are naturally occurring [26].

A. Carr et al. describes a murine IgG monoclonal antibody against NGcGM3 designated as 14F7 ([27], the content of which is incorporated herein by reference in its entirety for all purposes), which specifically and strongly recognizes this sialic acid with a nanomolar affinity range ([28,29], the content of each of which is incorporated herein by reference in its entirety for all purposes), and 14F7 selectively binds NGcGM3 rather than NAcGM3, which is naturally expressed on normal tissues and differs from NGcGM3 only in a single oxygen atom in the N-acetyl moiety.

Modified 14F7 antibody or its scFv fragments are also described in Rojas et al [38] and European Patent Application EP1623997, the content of each of which is incorporated herein by reference in its entirety for all purposes.

According to the present invention, targeting NGcGM3 expressing tumors with a CAR built with the scFv of 14F7 antibody (as well as humanized variants thereof [38]) has demonstrated therapeutic efficacy. Extensive in vitro characterization of different CAR T cells targeting NGcGM3, including cytotoxicity and cytokine production, as well as pre-clinical evaluation of the CAR T cells in syngeneic and xenograft models are described in the Examples section below.

### Definitions

The term “chimeric antigen receptor” or “CAR” as used herein is defined as a cell-surface receptor comprising an extracellular target-binding domain (e.g., an anti-NGcGM3 binding domain), a transmembrane domain, and an endodomain, comprising a signalling domain and optionally at least one co-stimulatory signaling domain (referred to also as intracellular (IC) domain herein), all in a combination that is not naturally found together on a single protein. This particularly includes receptors wherein the extracellular domain and the cytoplasmic domain are not naturally found together on a single receptor protein. The chimeric antigen receptors of the present invention are intended primarily for use with lymphocytes such as T cells and natural killer (NK) cells.

The terms “T cell” and “T lymphocyte” are interchangeable and used synonymously herein. As used herein, T cells include thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs are typically transcription factor Foxp3-positive CD4+ T cells and can also include transcription factor Foxp3-negative regulatory T cells that are IL-10-producing CD4+ T cells.

As used herein, the term “antigen” refers to any agent (e.g., protein, peptide, polysaccharide, glycoprotein, glycolipid (e.g., NGcGM3), nucleic acid, portions thereof, or combinations thereof) or molecule capable of being bound by a T-cell receptor or an antibody. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide (e.g., NGcGM3). Such a biological sample can include, but is not limited to, a tissue sample, a tumor sample, a cell or a fluid with other biological components, organisms, subunits of proteins/antigens, killed or inactivated whole cells or lysates.

“Host cells” of the present invention include T cells and natural killer cells that contain the DNA or RNA sequences encoding the CAR and express the CAR on the cell surface. Host cells may be used for enhancing T cell activity, natural killer cell activity, treatment of cancer, and treatment of autoimmune disease.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be the to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” refers to the addition of extra genetic material in the form of DNA or RNA into a cell.

As used herein, the term “derivative” or “derived from” in the context of proteins or polypeptides (e.g., CAR constructs or domains thereof) refer to: (a) a polypeptide with at least 40%, 45%, 50%, 55%, 60%, 65%, 70%, 75%, 80%, 85%, 90%, 95%, 98% or 99% sequence identity to the polypeptide it is a derivative of; (b) a polypeptide encoded by a nucleotide sequence with at least 40%, 45%, 50%, 55%, 60%, 65%, 70%, 75%, 80%, 85%, 90%, 95%, 98% or 99% sequence identity to a nucleotide sequence encoding the polypeptide it is a derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative of; (d) a polypeptide encoded by nucleic acids can hybridize under high, moderate or typical stringency hybridization conditions to nucleic acids encoding the polypeptide it is a derivative of; (e) a polypeptide encoded by a nucleotide sequence that can hybridize under high, moderate or typical stringency hybridization conditions to a nucleotide sequence encoding a fragment of the polypeptide, it is a derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wis.). Information regarding hybridization conditions (e.g., high, moderate, and typical stringency conditions) have been described, see, e.g., U.S. Patent Application Publication No. US 2005/0048549 (e.g., paragraphs 72-73).

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin, which is incorporated herein by reference in its entirety for all purposes.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric Antigen Receptors

In one aspect is provided a chimeric antigen receptor (CAR). The CAR may comprise an anti-NGcGM3 binding domain, an endodomain (ED), and a transmembrane (TM) domain. The endodomain may comprise a signalling domain. The transmembrane domain is typically disposed between the endodomain and the anti-NGcGM3 binding domain. In certain embodiments, the NGcGM3 is associated with a cancer cell or tumor cell.

The endodomain may comprise an intracellular (IC) domain interposed between the TM domain and the signalling domain. In some embodiments, the IC domain is derived from DAP10, DAP12, Fc epsilon receptor I gamma chain (FCER1G), FcR beta CD3-delta, CD3-epsilon, CD3-gamma, CD3-zeta, CD226, CD66d, CD79A, or CD79B. In certain embodiments, the IC domain may comprise the amino acid sequence of SEQ ID NO:54.

The anti-NGcGM3 binding domain may comprise an antigen-binding polypeptide, a receptor, or a natural ligand for a target cell antigen or receptor. The anti-NGcGM3 binding domain may comprise an antigen-binding polypeptide. Exemplary antigen-binding polypeptides include, but are not limited to, antibodies and antibody fragments. For example, the antigen-binding polypeptide can be a murine antibody, a rabbit antibody, a human antibody, a humanized antibody, a single chain variable fragment (scFv), a camelid antibody variable domain, a humanized version of a camelid antibody variable domain, a shark antibody variable domain, a humanized version of a shark antibody variable domain, a single domain antibody variable domain, a nanobody (VHHs), and a camelized antibody variable domain.

The TM domain may be derived from CD8, CD8a, CD4, CD3-zeta, CD3-epsilon, CD28, CD45, CD4, CD5, CD7, CD9, CD16, CD22, CD33, CD37, CD40, CD64, CD80, CD86, CD134 (OX-40), CD137, CD154, DAP10, or DAP12. In certain embodiments, the TM domain may comprise the amino acid sequence SEQ ID NO:52.

The signalling domain may be derived from DAP10, DAP12, Fc epsilon receptor I gamma chain (FCER1G), FcR beta CD3-delta, CD3-epsilon, CD3-gamma, CD3-zeta, CD226, CD66d, CD79A, or CD79B. In some embodiments, the signalling domain is derived from CD3-zeta. In certain embodiments, the signalling domain may comprise the amino acid sequence of SEQ ID NO:55.

The endodomain may comprise more than one signalling domain. For example, the endodomain may comprise two signalling domains.

In some embodiments, the CAR further comprises one or more additional polypeptide sequences. Exemplary additional polypeptide sequences include, but are not limited to, signal sequences, epitope tags, and polypeptides that produce a detectable signal.

The antigen-binding domain may comprise a linker. In certain embodiments, the linker may comprise the amino acid sequence of SEQ ID NO:16. Linkers are described in greater detail below.

The CAR may comprise a hinge domain interposed between the anti-NGcGM3 binding domain and the TM domain. The hinge domain may be an immunoglobulin hinge region. For example, the hinge region may be derived from CD8 or CD8-alpha. In certain embodiments, the hinge domain may comprise the amino acid sequence of SEQ ID NO:53. Hinge domains are described in greater detail below.

In some embodiments, the CAR includes a leader sequence. In certain embodiments, the leader sequence comprises the amino acid sequence SEQ ID NO:56.

### Leader Sequence

In one aspect, the CAR further comprises a leader sequence at the N-terminus of the extracellular antigen binding domain. The leader sequence is optionally cleaved from the antigen binding domain during cellular processing and localization of the CAR to the cellular membrane. In various embodiments, the leader sequence comprises an N-terminal CD8-alpha signal peptide (SEQ ID NO:56) for membrane targeting of the CAR. Signal peptides function to prompt a cell to translocate the protein to the cellular membrane.

In some embodiments, the leader sequence comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:56. In some embodiments, the leader sequence is encoded by a polynucleotide molecule having a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:70.

In various embodiments, the leader sequence comprises a leader sequence that is derived from human immunoglobulin heavy chain variable region. In various embodiments, the leader sequence comprises an amino acid sequence comprising, consisting of, or consisting essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 72 and/or is encoded by a nucleotide sequence comprising, consisting of, or consisting essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 71.

In some embodiments, the leader sequence comprises a leader sequence that is derived from the CD4 signal peptide. In various embodiments, the leader sequence comprises an amino acid sequence comprising, consisting of, or consisting essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 73.

N-terminal signal sequences can mediate targeting of nascent secretory and membrane proteins to the endoplasmic reticulum (ER) in a signal recognition particle (SRP)-dependent manner. Signal sequences may have a tripartite structure, consisting of a hydrophobic core region (h-region) flanked by an n- and c-region. The latter may contain a signal peptidase (SPase) consensus cleavage site. Usually, signal sequences are cleaved off co-translationally so that signal peptides and mature proteins are generated.

### Anti-NGcGM3 Binding Domain

In various embodiments, the NGcGM3 binding domain comprises an anti-NGcGM3 heavy chain variable domain sequence comprising: a heavy chain complementary determining region 1 (HC CDR1) sequence SYWIH (SEQ ID NO:3), a heavy chain complementary determining region 2 (HC CDR2) sequence YIDPATAYTESNQKFKD (SEQ ID NO:5), and a heavy chain complementary determining region 3 (HC CDR3) sequence ESPRLRRGIYYYAMDY (SEQ ID NO:7).

In various embodiments the anti-NGcGM3 binding domain comprises an anti-NGcGM3 heavy chain variable domain sequence comprising: a heavy chain complementary determining region 1 (HC CDR1) sequence SYWIH (SEQ ID NO:3), a heavy chain complementary determining region 2 (HC CDR2) sequence YIDPATAYTESNQKFKD (SEQ ID NO:5), a heavy chain complementary determining region 3 (HC CDR3) sequence ESPRLRRGIYYYAMDY (SEQ ID NO:7) and a framework region 1 (FR1) sequence QVQLQQSGASMKMSCRASGYSFT (SEQ ID NO:2), a framework region 2 (FR2) sequence WLKQRPDQGLEWIG (SEQ ID NO:4), a framework region 3 (FR3) sequence KAILTADRSSNTAFMYLNSLTSEDSAVYYCAR (SEQ ID NO:6), and a framework region 4 (FR4) sequence WGQGTSVTVSS (SEQ ID NO:8).

In various embodiments, the anti-NGcGM3 binding domain comprises an anti-NGcGM3 heavy chain variable domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:1.

In various embodiments, the NGcGM3 binding domain comprises an anti-NGcGM3 light chain variable domain sequence comprising a light chain complementarity determining region 1 (LC CDR1) sequence TGTSSDVGGYNHVS (SEQ ID NO:18), RASQSISSFLN (SEQ ID NO:25), or QGDSLRSYYAS (SEQ ID NO:32).

In some embodiments, the NGcGM3 binding domain comprises an anti-NGcGM3 light chain variable domain sequence comprising a light chain complementarity determining region 2 (LC CDR2) sequence DVSKRPS (SEQ ID NO:20), AASNLQS (SEQ ID NO:27), or GKNNRPS (SEQ ID NO:34).

In various embodiments, the NGcGM3 binding domain comprises an anti-NGcGM3 light chain variable domain sequence comprising a light chain complementarity determining region 3 (LC CDR3) sequence SSYAGSNNLVF (SEQ ID NO:22), QQGYTTPLTF(SEQ ID NO:29), NSRDSSGNHVVF (SEQ ID NO:36).

In certain embodiments, the anti-NGcGM3 binding domain comprises an anti-NGcGM3 light chain variable domain sequence comprising: i) a light chain complementary determining region 1 (LC CDR1) sequence TGTSSDVGGYNHVS (SEQ ID NO:18), a light chain complementary determining region 2 (LC CDR2) sequence DVSKRPS (SEQ ID NO:20), and a light chain complementary determining region 3 (LC CDR3) sequence SSYAGSNNLVF (SEQ ID NO:22), or ii) a light chain complementary determining region 1 (LC CDR1) sequence RASQSISSFLN (SEQ ID NO:25), a light chain complementary determining region 2 (LC CDR2) sequence AASNLQS (SEQ ID NO:27), and a light chain complementary determining region 3 (LC CDR3) sequence QQGYTTPLTF (SEQ ID NO:29), or iii) a light chain complementary determining region 1 (LC CDR1) sequence QGDSLRSYYAS (SEQ ID NO:32), a light chain complementary determining region 2 (LC CDR2) sequence GKNNRPS (SEQ ID NO:34), and a light chain complementary determining region 3 (LC CDR3) sequence NSRDSSGNHVVF (SEQ ID NO:36).

In certain embodiments, the anti-NGcGM3 binding domain comprises an anti-NGcGM3 light chain variable domain sequence comprising: i) a light chain complementary determining region 1 (LC CDR1) sequence TGTSSDVGGYNHVS (SEQ ID NO:18), a light chain complementary determining region 2 (LC CDR2) sequence DVSKRPS (SEQ ID NO:20), a light chain complementary determining region 3 (LC CDR3) sequence SSYAGSNNLVF (SEQ ID NO:22), a framework region 1 (FR1) sequence QSVVTQPPSASGGPGQSLTISC (SEQ ID NO:17), a framework region 2 (FR2) sequence WYQQHPGKAPKLMIY (SEQ ID NO:19), a framework region 3 (FR3) sequence GVPHRFSGSKSGNTASLTVSGLQAEDEAVYYC (SEQ ID NO:21), and a framework region 4 (FR4) sequence GGGTKVTVL (SEQ ID NO:23), or ii) a light chain complementary determining region 1 (LC CDR1) sequence RASQSISSFLN (SEQ ID NO:25), a light chain complementary determining region 2 (LC CDR2) sequence AASNLQS (SEQ ID NO:27), a light chain complementary determining region 3 (LC CDR3) sequence QQGYTTPLTF (SEQ ID NO:29), a framework region 1 (FR1) sequence DIQMTQTPSSLSASVGDRVTITC (SEQ ID NO:24), a framework region 2 (FR2) sequence WYQQKPGKAPKLLIY (SEQ ID NO:26), a framework region 3 (FR3) sequence GVPSRFSGRGSGTDFTLTISSLQPEDFAAYYC (SEQ ID NO:28), and a framework region 4 (FR4) sequence GQGTKLE (SEQ ID NO:30), or iii) a light chain complementary determining region 1 (LC CDR1) sequence QGDSLRSYYAS (SEQ ID NO:32), a light chain complementary determining region 2 (LC CDR2) sequence GKNNRPS (SEQ ID NO:34), a light chain complementary determining region 3 (LC CDR3) sequence NSRDSSGNHVVF (SEQ ID NO:36), a framework region 1 (FR1) sequence SSELTQDPAVSVALGQTVRITC (SEQ ID NO:31), a framework region 2 (FR2) sequence WYQQKPGQAPVLVIY (SEQ ID NO:33), a framework region 3 (FR3) sequence GIPDRFSGSSSGNTASLTITGAQAEDEADYYC (SEQ ID NO:35), and a framework region 4 (FR4) sequence GGGTKLTVL (SEQ ID NO:37).

In various embodiments, the anti-NGcGM3 binding domain comprises an anti-NGcGM3 light chain variable domain comprising an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:9.

In various embodiments, the anti-NGcGM3 binding domain comprises an anti-NGcGM3 light chain variable domain comprising an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:10.

In various embodiments, the anti-NGcGM3 binding domain comprises an anti-NGcGM3 light chain variable domain comprising an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:11.

In various embodiments, the CAR of the invention comprises a linker disposed between the heavy chain variable domain and the light chain variable domain of the anti-NGcGM3 binding domain. A linker may be derived from all or part of naturally occurring molecules. Alternatively, the linker may be a synthetic sequence that corresponds to a naturally occurring linker sequence, or may be an entirely synthetic linker sequence. In some embodiments, the linker is a Gly/Ser linker and comprises the amino acid sequence (Gly-Gly-Gly-Ser)n (SEQ ID NO: 75), where n is a positive integer equal to or greater than 1. For example, n=1, n=2, n=3, n=4, n=5 and n=6, n=7, n=8, n=9 and n=10. In some embodiments, the linkers include, but are not limited to, (Gly4 Ser)4 (SEQ ID NO:76) or (Gly4 Ser)3 (SEQ ID NO:77). In another embodiment, the linkers include multiple repeats of (Gly2Ser), (GlySer) or (Gly3Ser) (SEQ ID NO:78). In one embodiment, the linker is GSTSGSGKPGSGEGSTKG (SEQ ID NO: 79). Also included within the scope of the invention are linkers described in WO2012/138475, the content of which is incorporated herein by reference in its entirety for all purposes.

In various embodiments, the CAR of the invention comprises a linker with an amino acid sequence comprising, consisting of, or consisting essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:16.

In some embodiments, the anti-NGcGM3 binding domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:67. In some embodiments, the anti-NGcGM3 binding domain is encoded by a polynucleotide molecule having a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:57.

In some embodiments, the anti-NGcGM3 binding domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:68. In some embodiments, the anti-NGcGM3 binding domain is encoded by a polynucleotide molecule having a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:59.

In some embodiments, the anti-NGcGM3 binding domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:69. In some embodiments, the anti-NGcGM3 binding domain is encoded by a polynucleotide molecule having a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:66.

### Hinge Domain

In various embodiments, a hinge domain can be used to provide more flexibility and accessibility for the antigen-binding moiety. A hinge domain may comprise up to 300 amino acids, preferably 10 to 100 amino acids and most preferably 25 to 50 amino acids. A hinge domain may be derived from all or part of naturally occurring molecules, such as from all or part of the extracellular region of CD8, CD4 or CD28, or from all or part of an antibody constant region. Alternatively, the hinge domain may be a synthetic sequence that corresponds to a naturally occurring hinge domain sequence, or may be an entirely synthetic hinge domain sequence. Non-limiting examples of a hinge domains which may be used in accordance to the invention include a part of human CD8 alpha chain, partial extracellular domain of CD28, FcγRllla receptor, IgG, IgM, IgA, IgD, IgE, an Ig hinge, or functional fragment thereof. In certain embodiments, additional amino acids are added to the hinge domain to ensure that the anti-NGcGM3 binding domain is at an optimal distance from the TM domain. In certain embodiments, when the hinge domain is derived from an Ig, the hinge domain may be mutated to prevent Fc receptor binding.

In some embodiments, the hinge domain of the CAR of the invention is derived from CD28. Cluster of differentiation 28 (CD28) is a co-stimulatory protein (a T cell costimulatory receptor). CD28 is the only B7 receptor constitutively expressed on naive T cells. CD28 is critical for T cell-dependent antibody responses. CD28 is a TM cell surface glycoprotein belonging to Ig superfamily.

In various embodiments, the hinge domain comprises a hinge having an amino acid sequence comprising, consisting of, or consisting essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:53 and/or is encoded by a gene comprising, consisting of, or consisting essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:51.

In certain embodiments, the hinge domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the hinge domain comprises the CH1, CH2, CH3 and/or hinge domain of the immunoglobulin. In certain embodiments, the hinge domain comprises the core hinge domain of the immunoglobulin. The term “core hinge” can be used interchangeably with the term “short hinge” (a.k.a “SH”). Non-limiting examples of suitable hinge domains are the core immunoglobulin hinge domain sequences listed in Table 1 (see also Wypych et al., JBC 2008 283(23): 16194-16205, which is incorporated herein by reference in its entirety for all purposes). In certain embodiments, the hinge domain is a fragment of the immunoglobulin hinge domain.

TABLE 1
Amino Acid Sequence of Core Hinge Regions of IgG
Immunoglobulins

| IgG Subtype | Core Hinge Domain Sequence |
|---|---|
| IgG1 | EPKSCDKTHTCPPCP (SEQ ID NO: 12) |
| IgG2 | ERKCCVECPPCP (SEQ ID NO: 13) |
| IgG3 | ELKTPLGDTTHTCPRCP(EPKSCDTPPPCPRCP)₃ |
| | (SEQ ID NO: 14) |
| IgG4 | ESKYGPPCPSCP (SEQ ID NO: 15) |

In certain embodiments, the hinge domain comprises an IgG1 hinge, or a variant thereof. In certain embodiments, the hinge domain comprises the core hinge domain of IgG1 or a variant thereof. In certain embodiments, the hinge domain comprises an IgG2 hinge, or a variant thereof. In certain embodiments, the hinge domain comprises the core hinge structure of IgG2 or a variant thereof.

### Transmembrane (TM) Domain

In certain embodiments, the TM domain is fused in frame between the anti-NGcGM3 binding domain and the ED. The TM domain may be derived from a protein contributing to the anti-NGcGM3 binding domain, the protein contributing the signalling domain or a co-signalling domain, or by a totally different protein. In some instances, the TM domain can be selected or modified by amino acid substitution, deletions, or insertions to minimize interactions with other members of the CAR complex. In some instances, the TM domain can be selected or modified by amino acid substitution, deletions, or insertions to avoid-binding of proteins naturally associated with the TM domain. In certain embodiments, the TM domain includes additional amino acids to allow for flexibility and/or optimal distance between the domains connected to the TM domain.

The TM domain may be derived either from a natural or from a synthetic source. Where the source is natural, the domain may be derived from any membrane-bound or TM protein. Non-limiting examples of TM domains of particular use in this invention may be derived from (i.e. comprise at least the TM region(s) of) the alpha, beta, or zeta chain of the T-cell receptor, CD28, CD3 epsilon, CD45, CD4, CD5, CD8, CD9, CD16, CD22, CD33, CD37, CD40, CD64, CD80, CD86, CD134, CD137, CD154. Alternatively, the TM domain may be synthetic, in which case it will comprise predominantly hydrophobic residues such as leucine and valine. For example, a triplet of phenylalanine, tryptophan and/or valine can be found at each end of a synthetic TM domain. In some embodiments, the TM domain of the CAR of the invention is derived from CD28.

In certain embodiments, it will be desirable to utilize the TM domain of the zeta, eta, or Fc epsilon receptor I gamma chains which contain a cysteine residue capable of disulfide bonding, so that the resulting chimeric protein will be able to form disulfide linked dimers with itself, or with unmodified versions of the zeta, eta, or Fc epsilon receptor I gamma chains or related proteins. In some instances, the TM domain will be selected or modified by amino acid substitution to avoid-binding of such domains to the TM domains of the same or different surface membrane proteins to minimize interactions with other members of the receptor complex. In other cases, it will be desirable to employ the TM domain of zeta, eta, or Fc epsilon receptor I gamma and beta, MB1 (Igα), B29 or CD3-gamma, zeta, or eta, in order to retain physical association with other members of the receptor complex.

In certain embodiments, the TM domain in the CAR of the invention is derived from the CD8-alpha TM domain.

In some embodiments, the TM region comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:52. In some embodiments, the TM region of the CAR is encoded by a DNA molecule with a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:49.

### IC Domain

In some embodiments, the IC domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:54. In some embodiments, the IC domain of the CAR is encoded by a DNA molecule with a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:48.

### Signalling Domain

In certain embodiments, the ED comprises one or more signalling domains.

In certain embodiments, the signalling domains can be in any order. The ED, which comprises the signaling domain of the CAR of the invention, is typically responsible for activation of at least one of the normal effector functions of the lymphocyte in which the CAR has been placed in. The term “effector function” refers to a specialized function of a cell. Effector function of a T cell, for example, may be cytolytic activity or helper activity including the secretion of cytokines. Thus, the term “signalling domain” refers to the portion of a protein which transduces the effector function signal and directs the cell to perform a specialized function. While usually the entire signaling domain is present, in many cases it is not necessary to use the entire chain. To the extent that a truncated portion of the signalling domain is used, such truncated portion may be used in place of the intact chain as long as it transduces the effector function signal. The term signalling domain is thus meant to include any truncated portion of the signaling domain sufficient to transduce the effector function signal.

Non-limiting examples of signaling domains which can be used in the CARs of the invention include, e.g., signalling domains derived from DAP10, DAP12, Fc epsilon receptor I gamma chain (FCER1G), FcR beta, CD3-delta, CD3-epsilon, CD3-gamma, CD3-zeta, CD5, CD22, CD226, CD66d, CD79A, and CD79B.

In various embodiments the signalling domain is derived from CD3-zeta. CD3-zeta is a homodimer-forming type 1 TM protein and is part of the T-cell antigen receptor (TCR-CD3) complex along with TCR-alpha-beta, CD3-gamma-epsilon, and CD3-delta-epsilon dimers expressed on the surface of T cells. CD3-zeta possesses a small extracellular part, a TM region, and a long cytoplasmic part that contains three immunoreceptor tyrosine-based activation motifs (ITAMs), which correspond to the six tyrosines that get phosphorylated upon antigen binding to the extracellular part of TCRαβ. Phosphorylation subsequently activates several downstream signalling cascades resulting in activation of a T cell (Deswal S., Schamel W. W. A. (2012) CD3-zeta. In: Choi S. (eds) Encyclopedia of Signalling Molecules. Springer, New York, N.Y., the contents of which are incorporated herein in their entirety for all purposes). In some embodiments, the signalling domain comprises the cytoplasmic domain of CD3-zeta or a portion thereof. In certain embodiments, the signaling domain in the CAR of the invention is designed to comprise the signalling domain of CD3-zeta.

In certain embodiments, the CARs of the invention can include additional signaling domains. Non-limiting signaling domains include, but are not limited to, 4-1BB (CD137), CD28, ICOS, CD134 (OX-40), BTLA, CD27, CD30, GITR, CD226, CD40, and HVEM.

In some embodiments, the signalling domain of the CAR comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:55. In some embodiments, the IC signalling region of the CAR is encoded by a DNA molecule with a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:50.

### Accessory Genes

In addition to the CAR construct, the CAR may further comprise an accessory gene that encodes an accessory peptide. Examples of accessory genes can include a transduced host cell selection marker, an in vivo tracking marker, a cytokine, a suicide gene, or some other functional gene.

In certain embodiments, the CAR comprises at least one accessory gene. In certain embodiments, the CAR comprises one accessory gene. In other embodiments, the CAR comprises two accessory genes. In yet another embodiment, the CAR comprises three accessory genes.

In certain embodiments, the accessory gene is tCD19. In certain embodiments, the tCD19 can be used as a tag. For example, expression of tCD19 can help determine transduction efficiency. In certain embodiments, the CAR comprises the tCD19 construct. In certain embodiments, the CAR does not include the tCD19 construct. In certain embodiments, the tCD19 can be replaced with a functional accessory gene to enhance the effector function of the CAR (e.g., NGcGM3-CAR) containing host cells. In certain embodiments, the functional accessory gene can increase the safety of the CAR.

In some embodiments, the accessory gene is a fluorescent protein, such as, but not limited to, green fluorescent protein (GFP), yellow fluorescent protein (YFP), or cyan fluorescent protein (CFP).

Non-limiting examples of classes of accessory genes that can be used to increase the effector function of CAR containing host cells, include i) secretable cytokines (e.g., but not limited to, IL-7, IL-12, IL-15, IL-18), ii) membrane bound cytokines (e.g., but not limited to, IL-15), iii) chimeric cytokine receptors (e.g., but not limited to, IL-2/IL-7, IL-4/IL-7), iv) constitutive active cytokine receptors (e.g., but not limited to, C7R), v) dominant negative receptors (DNR; e.g., but not limited to TGFRII DNR), vi) ligands of costimulatory molecules (e.g., but not limited to, CD80, 4-1BBL), vii) antibodies, including fragments thereof and bispecific antibodies (e.g., but not limited to, bispecific T-cell engagers (BiTEs)), or vii) a second CAR.

In certain embodiments, the functional accessory gene can be a suicide gene. A suicide gene is a recombinant gene that will cause the host cell that the gene is expressed in to undergo programmed cell death or antibody mediated clearance at a desired time. Suicide genes can function to increase the safety of the CAR. In another embodiment, the accessory gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105(11): 4247-4254; US Publ. No. 2011/0286980, each of which are incorporated herein by reference in their entirety for all purposes)).

When two or more accessory genes are used, they can be separated by a separation sequence (e.g., a 2A sequence) using a combination of the classes of molecules listed above (e.g., CAR-2A-CD20-2A-IL15). In addition, the use of two separation sequences (e.g., 2A sequences) would allow the expression of TCR (e.g., CAR-2A-TCRα-2A-TCRβ). In the constructs with a CAR and two or three accessory genes, the order of the CAR and the second or third transgene could be switched.

A “separation sequence” refers to a peptide sequence that can cause a ribosome to release a growing polypeptide chain that is being synthesized without dissociation from the mRNA. In this respect, the ribosome continues translating and therefore produces a second polypeptide. Non-limiting examples of separation sequences includes T2A (EGRGSLLTCGDVEENPGP (SEQ ID NO:65) or GSGEGRGSLLTCGDVEENPGP (SEQ ID NO:38)); the foot and mouth disease virus (FMDV) 2A sequence (GSGSRVTELLYRMKRAETYCPRPLLAIHPTEARHKQKIVAPVKQLLNFDLLKLAGDVES NPGP (SEQ ID NO:39)); Sponge (Amphimedon queenslandica) 2A sequence (LLCFLLLLLSGDVELNPGP (SEQ ID NO:40), or HHFMFLLLLLAGDIELNPGP (SEQ ID NO:41)); acorn worm (Saccoglossus kowalevskii) 2A sequence (WFLVLLSFILSGDIEVNPGP (SEQ ID NO:42)); amphioxus (Branchiostoma floridae) 2A sequence (KNCAMYMLLLSGDVETNPGP (SEQ ID NO:43), or MVISQLMLKLAGDVEENPGP (SEQ ID NO:44)); porcine teschovirus-1 2A sequence (GSGATNFSLLKQAGDVEENPGP (SEQ ID NO:45)); and equine rhinitis A virus 2A sequence (GSGQCTNYALLKLAGDVESNPGP (SEQ ID NO:46)). In some embodiments, the separation sequence is a naturally occurring or synthetic sequence. In certain embodiments, the separation sequence includes the 2A consensus sequence D-X-E-X-NPGP (SEQ ID NO: 74), in which X is any amino acid residue.

### Exemplary CAR Sequences

In some embodiments, the CAR may comprise, consist of, or consist essentially of an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:61. In some embodiments, the CAR is encoded by a DNA molecule that comprises, consists of, or consists essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:64.

In some embodiments, the CAR comprises, consist of, or consist essentially of an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:62. In some embodiments, the CAR is encoded by a DNA molecule that comprises, consists of, or consists essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:58.

In some embodiments, the CAR comprises, consist of, or consist essentially of an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:63. In some embodiments, the CAR is encoded by a DNA molecule that comprises, consists of, or consists essentially of a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:60.

### Nucleic Acid Molecules

In one aspect is provided a nucleic acid molecule comprising a nucleotide sequence encoding any chimeric antigen receptor (CAR) described herein.

In a specific embodiment, the nucleic acid molecule may comprise, or consist of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:64.

In a specific embodiment, the nucleic acid molecule may comprise, or consist of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:58.

In a specific embodiment, the nucleic acid molecule may comprise, or consist of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO:60.

In various embodiments, the nucleotide sequence encoding the CAR is operably linked to a promoter. In various embodiments the promoter is a PGK promoter. In various embodiments, the promoter is a T lymphocyte-specific promoter or an NK cell-specific promoter. In various embodiments, the nucleic acid molecule is a DNA molecule. In various embodiments, the nucleic acid molecule is an RNA molecule.

### Vectors

In one aspect is provided a recombinant vector comprising any nucleic acid molecule described herein, or any nucleic acid encoding any polypeptide described herein. In some embodiments, the recombinant vector is a viral vector. The vector may be a retroviral vector, a lentiviral vector, an adenoviral vector, an adeno-associated virus vector, an alphaviral vector, a herpes virus vector, or a vaccinia virus vector. In some embodiments, the vector is a lentiviral vector.

### Host Cells

In another aspect is provided an isolated host cell comprising any CAR described herein. The isolated host cell may comprise any nucleic acid molecule described herein. The isolated host cell may comprise any vector described herein. The host cell may be a mammalian cell. Exemplary host cells include, but are not limited to, cytotoxic cells, T cells, stem cells, progenitor cells, and cells derived from a stem cell or a progenitor cell. The T cell may be a T-helper cell, a cytotoxic T-cell, a T-regulatory cell (Treg), or a gamma-delta T cell. The cytotoxic cell may be a cytotoxic T cell or a natural killer (NK) cell. The host cell may be activated ex vivo and/or expanded ex vivo. The host cell may be an allogeneic cell. The host cell may be an autologous cell. The host cell may be isolated from a subject having a disease. In various embodiments, the subject is human.

Also provided is a method for producing any of the above host cells. The method comprises genetically modifying the cell with any nucleic acid molecule or any vector described herein. The genetic modification may be conducted ex vivo. The method may further comprise activation and/or expansion of the cell ex vivo.

The polypeptides disclosed herein, or nucleic acids encoding such, may be introduced into the host cells using transfection and/or transduction techniques known in the art. The nucleic acid may be integrated into the host cell DNA or may be maintained extrachromosomally. The nucleic acid may be maintained transiently or may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In certain embodiments, retroviral vectors are transduced by packaging the vectors into virions prior to contact with a cell. For example, a nucleic acid encoding a transmembrane polypeptide carried by a retroviral vector can be transduced into a cell through infection and pro virus integration.

In certain embodiments, the nucleic acid or viral vector is transferred via ex vivo transformation. Methods for transfecting vascular cells and tissues removed from an organism in an ex vivo setting are known to those of skill in the art. Thus, it is contemplated that cells or tissues may be removed and transfected ex vivo using the polynucleotides presented herein. In particular aspects, the transplanted cells or tissues may be placed into an organism. Thus, it is well within the knowledge of one skilled in the art to isolate antigen-presenting cells (e.g., T-cells or NK cells) from an animal (e.g., human), transfect the cells with the expression vector and then administer the transfected or transformed cells back to the animal.

In certain embodiments, the nucleic acid or viral vector is transferred via injection. In certain embodiments, a polynucleotide is introduced into an organelle, a cell, a tissue or an organism via electroporation. In certain embodiments, a polynucleotide is delivered into a cell using DEAE-dextran followed by polyethylene glycol. In certain embodiments, the polynucleotides encode any of the first and second transmembrane polypeptides described herein, and are inserted into a vector or vectors. The vector is a vehicle into which a polynucleotide encoding a protein may be covalently inserted so as to bring about the expression of that protein and/or the cloning of the polynucleotide. Expression vectors have the ability to incorporate and express heterologous or modified nucleic acid sequences coding for at least part of a gene product capable of being transcribed in a cell. In most cases, RNA molecules are then translated into a protein.

Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well. An expression vector may comprise additional elements, for example, the expression vector may have two replication systems, thus allowing it to be maintained in two organisms, for example in human cells for expression and in a prokaryotic host for cloning and amplification. The expression vector may have additional sequence such as 6×-histidine, c-Myc, and FLAG tags which are incorporated into the expressed polypeptides. In various embodiments, the vectors are plasmid, autonomously replicating sequences, and transposable elements.

In certain embodiments, the nucleic acids encoding the transmembrane polypeptides of the present invention are provided in a viral vector. In certain embodiments, the viral vector is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

In certain embodiments, the present disclosure provides isolated host cells (e.g., T-cells) containing the vectors provided herein. The host cells containing the vector may be useful in expression or cloning of the polynucleotide contained in the vector.

### Pharmaceutical Compositions

In another aspect is provided a pharmaceutical composition comprising any host cell described herein, and a pharmaceutically acceptable carrier and/or excipient. Exemplary carriers include, but are not limited to, sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432, the content of which is incorporated herein by reference in its entirety for all purposes).

The pharmaceutical composition may be used in combination with other therapies. It is contemplated that when used to treat various diseases, the compositions and methods can be combined with other therapeutic agents suitable for the same or similar diseases. Also, two or more embodiments described herein may be also co-administered to generate additive or synergistic effects. When co-administered with a second therapeutic agent, the embodiment described herein and the second therapeutic agent may be simultaneously or sequentially (in any order). Suitable therapeutically effective dosages for each agent may be lowered due to the additive action or synergy.

As a non-limiting example, the methods described herein can be combined with other therapies that block inflammation (e.g., via blockage of ILL INFα/β, IL6, TNF, IL13, IL23, etc.).

In some embodiments, the compositions and methods disclosed herein are useful to enhance the efficacy of vaccines directed to tumors. Thus, the compositions and methods described herein can be administered to a subject either simultaneously with or before (e.g., 1-30 days before) a reagent (including but not limited to small molecules, antibodies, or cellular reagents) that acts to elicit an immune response (e.g., to treat cancer) is administered to the subject.

The compositions and methods described herein can be also administered in combination with an anti-tumor antibody or an antibody directed at a pathogenic antigen or allergen.

The compositions and methods described herein can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments described herein can be also combined with other treatments that possess the ability to modulate NKT function or stability, including but not limited to CD1d, CD1 d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e), in any of the aforementioned forms or formulations, alone or in combination with each other or other agents.

Therapeutic methods described herein can be combined with additional immunotherapies and therapies. For example, when used for treating cancer, NKT cells described herein can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors described herein include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In some embodiments, the inhibitors described herein can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

The present invention provides methods which comprise administering a pharmaceutical composition comprising any of the exemplary CAR described herein in combination with one or more additional therapeutic agents. Exemplary additional therapeutic agents that may be combined with or administered in combination with a CAR include, e.g., an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2, anti-ErbB3 or anti-ErbB4 antibody or small molecule inhibitor of ErbB2, ErbB3 or ErbB4 activity), an antagonist of EGFRvIII (e.g., an antibody that specifically binds EGFRvIII), a cMET antagonist (e.g., an anti-cMET antibody), an IGF1R antagonist (e.g., an anti-IGF1R antibody), a B-raf inhibitor (e.g., vemurafenib, sorafenib, GDC-0879, PLX-4720), a PDGFR-α inhibitor (e.g., an anti-PDGFR-α antibody), a PDGFR-β inhibitor (e.g., an anti-PDGFR-β antibody), a VEGF antagonist (e.g., a VEGF-Trap, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as H1H685P), a FOLH1 (PSMA) antagonist, a PRLR antagonist (e.g., an anti-PRLR antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin antibody), etc. Other agents that may be beneficially administered in combination with a CAR include cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors. The pharmaceutical compositions of the present invention may also be administered as part of a therapeutic regimen comprising one or more therapeutic combinations such as, but not limited to, “ICE”: ifosfamide (e.g., Ifex®), carboplatin (e.g., Paraplatin®), etoposide (e.g., Etopophos®, Toposar®, VePesid®, VP-16); “DHAP”: dexamethasone (e.g., Decadron®), cytarabine (e.g., Cytosar-U®, cytosine arabinoside, ara-C), cisplatin (e.g., Platinol®-AQ) “ESHAP”: etoposide (e.g., Etopophos®, Toposar®, VePesid®, VP-16), methylprednisolone (e.g., Medrol®), high-dose cytarabine, or cisplatin (e.g., Platinol®-AQ).

The present invention also includes therapeutic combinations comprising any of the antigen-binding molecules mentioned herein and an inhibitor of one or more of VEGF, Ang2, DLL4, EGFR, ErbB2, ErbB3, ErbB4, EGFRvIII, cMet, IGF1R, B-raf, PDGFR-α, PDGFR-β, FOLH1 (PSMA), PRLR, STEAP1, STEAP2, TMPRSS2, MSLN, CA9, uroplakin, or any of the aforementioned cytokines, wherein the inhibitor is an aptamer, an antisense molecule, a ribozyme, an siRNA, a peptibody, a nanobody or an antibody fragment (e.g., Fab fragment; F(ab′)2 fragment; Fd fragment; Fv fragment; scFv; dAb fragment; or other engineered molecules, such as diabodies, triabodies, tetrabodies, minibodies and minimal recognition units). The CAR may also be administered and/or co-formulated in combination with antivirals, antibiotics, analgesics, corticosteroids and/or NSAIDs. The antigen-binding molecules of the invention may also be administered as part of a treatment regimen that also includes radiation treatment and/or conventional chemotherapy.

Non-limiting examples of chemotherapeutic compounds which can be used in combination treatments include, for example, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphami de, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

The additional therapeutically active component(s) may be administered just prior to, concurrent with, or shortly after the administration of a CAR (for purposes of the present disclosure, such administration regimens are considered the administration of a CAR “in combination with” an additional therapeutically active component).

The present invention includes pharmaceutical compositions in which a CAR is co-formulated with one or more of the additional therapeutically active component(s) as described elsewhere herein.

### Therapeutic Uses

The present invention includes methods comprising administering to a subject in need thereof a therapeutic composition comprising a CAR as described herein. The therapeutic composition can comprise any of the CARs as disclosed herein and a pharmaceutically acceptable carrier or diluent. As used herein, the expression “a subject in need thereof” means a human or non-human animal that exhibits one or more symptoms or indicia of an infection (e.g., a subject suffering from a bacterial or viral infection, including any of those mentioned herein) cancer (e.g., a subject expressing a tumor or suffering from any of the cancers mentioned herein), an autoimmune disorder (e.g., a subject suffering from any of the autoimmune diseases or disorders mentioned herein), inflammatory diseases, or who otherwise would benefit from enhancement or suppression of T cell activity.

The anti-tumor responses of T cells after exposure to the CAR may be determined in xenograft tumor models. Tumors may be established using any human cancer cell line expressing the tumor associated antigen presented by the CAR. In order to establish xenograft tumor models, about 5×10⁶ viable cells, may be injected, e.g., s.c., into nude athymic mice using for example Matrigel (Becton Dickinson). The endpoint of the xenograft tumor models can be determined based on the size of the tumors, weight of animals, survival time and histochemical and histopathological examination of the cancer, using methods known to one skilled in the art.

According to certain aspects, a CAR may be used to treat a cancer in which the tumor cells express a tumor-associated antigen, for example, NGcGM3.

Specific cancers/tumors treatable by the methods and CARs of the present invention include, without limitation, various solid malignancies, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all typies including but not limited to EBV-associated Hodkin's and non-Hodgkin's lymphoma, all forms of post-transplant lymphomas including post-transplant lymphoproliferative disorder (PTLD), uterine cancer, renal cell carcinoma, hepatoma, hepatoblastoma, Cancers that may treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The present invention also includes methods for treating residual cancer in a subject. As used herein, the term “residual cancer” means the existence or persistence of one or more cancerous cells in a subject following treatment with an anti-cancer therapy.

The present invention also includes use of the CARs herein in the manufacture of a medicament for preventing, treating and/or ameliorating an infection, a cancer, or an autoimmune disorder (e.g., as discussed herein).

In one aspect is provided a method for stimulating elimination of a cell comprising an antigen in a subject in need thereof. The method comprises administering to the subject an effective amount of cytotoxic T cells or natural killer (NK) cells comprising any heterodimeric CAR described herein, wherein the anti-NGcGM3 binding domain of the CAR binds to the antigen.

The antigen may be a cancer cell associated antigen, an infection-associated antigen or an auto-antigen. The antigen may be a cancer cell associated antigen. The cancer cell associated antigen may be associated with a solid tumor. The antigen may be an infection-associated antigen. The antigen may be an auto-antigen. The antigen may be NGcGM3.

In another aspect is provided a method for stimulating elimination of a cell comprising NGcGM3. The method comprises administering to the subject an effective amount of cytotoxic T cells or natural killer (NK) cells comprising a CAR described herein.

In another aspect is provided a method for treating a cancer in a subject in need thereof. The method comprises administering to the subject a therapeutically effective amount of cytotoxic T cells or natural killer (NK) cells comprising any chimeric antigen receptor (CAR) described herein, wherein the extracellular anti-NGcGM3 binding domain of the CAR binds to an antigen associated with the cancer. The cancer may be from a solid tumor. The cancer may be carcinoma, melanoma, prostate cancer, sarcoma, glioma, astrocytoma, medulloblastoma, craniopharyngioma, ependymoma, pinealoma, hemangioblastoma, acoustic neuroma, oligodendroglioma, menangioma, neuroblastoma, or retinoblastoma. The cancer may be a leukemia or a lymphoma.

In another aspect is provided a method for treating an ovarian tumor in a subject in need thereof. The method comprises administering to the subject a therapeutically effective amount of cytotoxic T cells or natural killer (NK) cells comprising any CAR described herein.

The method may comprise a) isolating T cells or NK cells from the subject; b) genetically modifying the T cells or NK cells ex vivo with any nucleic acid molecule or any vector described herein. The T cells or NK cells may be expanded or activated before, after or during step (b). The genetically modified T cells or NK cells are introduced into the subject.

In various embodiments, the subject is human.

According to certain embodiments of the present invention, multiple doses of a CAR may be administered to a subject over a defined time course. The methods according to this aspect of the invention comprise sequentially administering to a subject multiple doses of a CAR of the invention. As used herein, “sequentially administering” means that each dose of a CAR is administered to the subject at a different point in time, e.g., on different days separated by a predetermined interval (e.g., hours, days, weeks or months). The present invention includes methods which comprise sequentially administering to the patient a single initial dose of a CAR, followed by one or more secondary doses of the CAR, and optionally followed by one or more tertiary doses of the CAR.

The terms “initial dose,” “secondary doses,” and “tertiary doses,” refer to the temporal sequence of administration of the CAR. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the CAR, but generally may differ from one another in terms of frequency of administration. In certain embodiments, however, the amount of a CAR contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of CAR which is administered to a patient prior to the administration of the very next dose in the sequence with no intervening doses.

The methods according to this aspect of the invention may comprise administering to a patient any number of secondary and/or tertiary doses of a CAR. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Examples

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

Example 1. Demonstrating that humanized versions of 14F7-derived CARs are stably expressed on both immortal and primary human T cells and the humanized versions of 14F7-derived CARs recognize an antigen-expressing human tumor cell line in vitro.

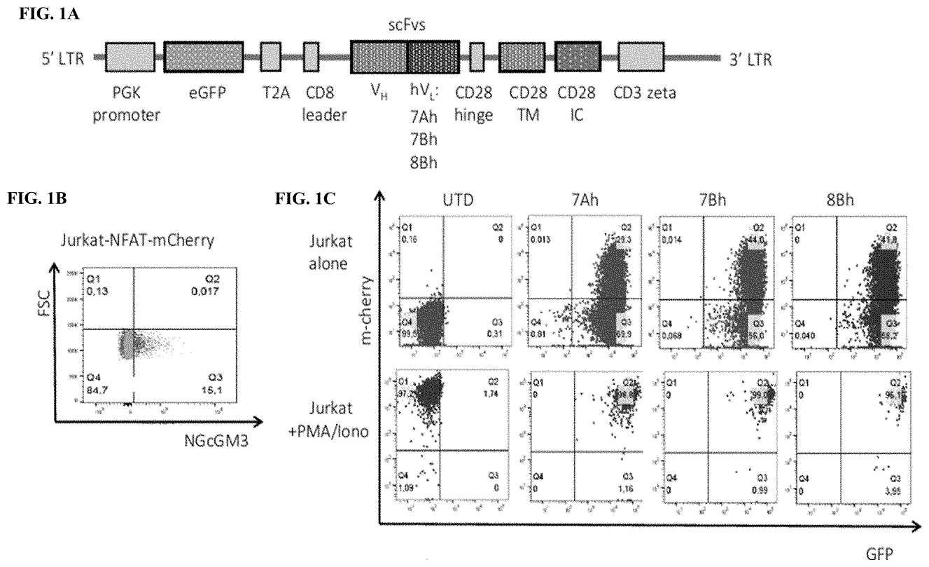

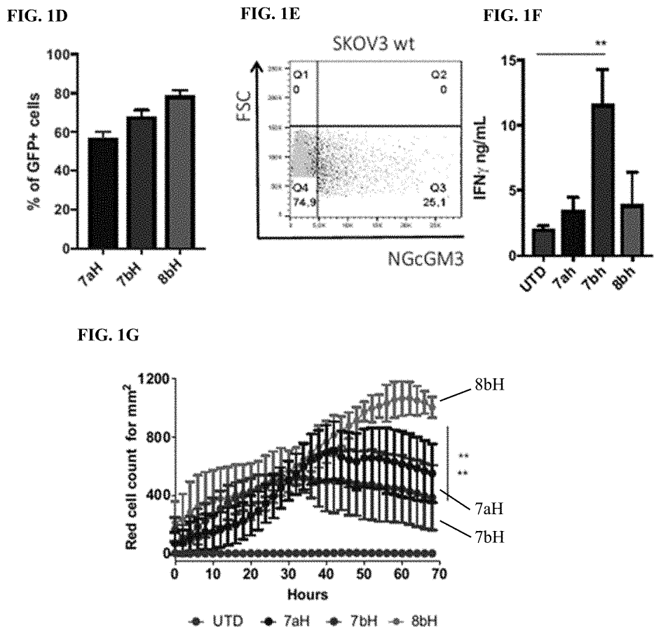

Three humanized versions of 14F7-derived CARs (variants of 14F7-derived CAR) were generated by cloning the VH chain and three humanized variants of the VL chain [VL 7Ah (SEQ ID NO:9), VL 7Bh (SEQ ID NO:10) and VL 8Bh (SEQ ID NO:11)] as previously described [38] in a pRRL-based lentiviral vector encoding a human CD8 leader sequence, a CD28-derived hinge domain, a TM domain, an IC domain and a CD3-signalling domain, to provide 7Ah (SEQ ID NO:61), 7Bh (SEQ ID NO:62), and 8Bh (SEQ ID NO:63) CARs each under the control of a phosphoglycerate kinase (PGK) promoter, in a bicistronic construct together with a reporter green fluorescent protein (GFP) (FIG. 1A). The human T lymphocytes cell line Jurkat was stably transduced to express the marker mCherry under the control of the nuclear factor of activated T cells promoter (NFAT) to prepare Jurkat-NFAT-mCherry cells. The Jurkat-NFAT-mCherry cells physiologically expressed NGcGM3 (15.1%) (FIG. 1B). Jurkat-NFAT-mCherry cells were transduced to express one of the 3 variants of 14F7-derived CAR [7Ah (SEQ ID NO:61), 7Bh (SEQ ID NO:62), and 8Bh (SEQ ID NO:63)]. Cells transduced to express one of the 3 variants of 14F7-derived CAR are referred to herein as 7Ah, 7Bh, and 8Bh cells for brevity. Untransduced Jurkat-NFAT-mCherry (UTD) cells were used as a control. Transduced Jurkat cells were kept in culture for 48h and demonstrated reciprocal antigen induced activation; whereas, UTD did not. Reciprocal antigen induced activation was demonstrated by simultaneous expression of mCherry and GFP indicating that the 7Ah, 7Bh, and 8Bh cells were activated against each other during culture. As a positive control of mCherry expression under control of NFAT, cells were treated with Phorbol 12-Myristate 13-Acetate/Ionomycin (PMA/Iono) (FIG. 1C).

Primary human T cells were isolated from a healthy donor apheresis, activated with anti-CD3/anti-CD28 dynabeads in presence of 50 IU/ml hIL2, and efficiently transduced (above 50%) with one of the 3 variants of 14F7-derivd CAR to prepare CAR T cells (as above, individually referred to as 7Ah, 7Bh, and 8Bh cells for brevity). Untransduced primary human T cells (UTD) were used as a negative control. Successful transduction was verified by expression of GFP evaluated using flow cytometry (FIG. 1D). Prior to all in vitro and in vivo experimentation, T cell groups were normalized for equal CAR cell-surface expression. Antigen-induced activation of the CAR T cells was tested in an in vitro assay against ovarian tumor cell line SKOV3 wild-type (wt.) expressing NGcGM3 (25% expression level was verified, FIG. 1E). Upon 24h co-culture of the CAR T cells with SKOV3 (a 24 h co-culture experiment), only the 7Bh cells demonstrated a statistically significant higher IFN release as compared to UTD (p=0.0035, student t test) (FIG. 1F). In a long-term co-culture experiment (68h), the three CAR T cells (7Ah, 7Bh, and 8Bh) recognized and efficiently killed target cells (SKOV3); whereas, UTD cells did not. Dead cell count/mm³ was performed using an IncuCyte instrument. In view of the above, 8Bh cells were the most efficient cells at killing target cells (SKOV3), as compared to 7Ah and 7Bh cells (FIG. 1G).

Example 2. Demonstrating that primary human T cells expressing humanized versions of 14F7-derived CARs efficiently control tumor growth of an in vivo model of ovarian cancer.

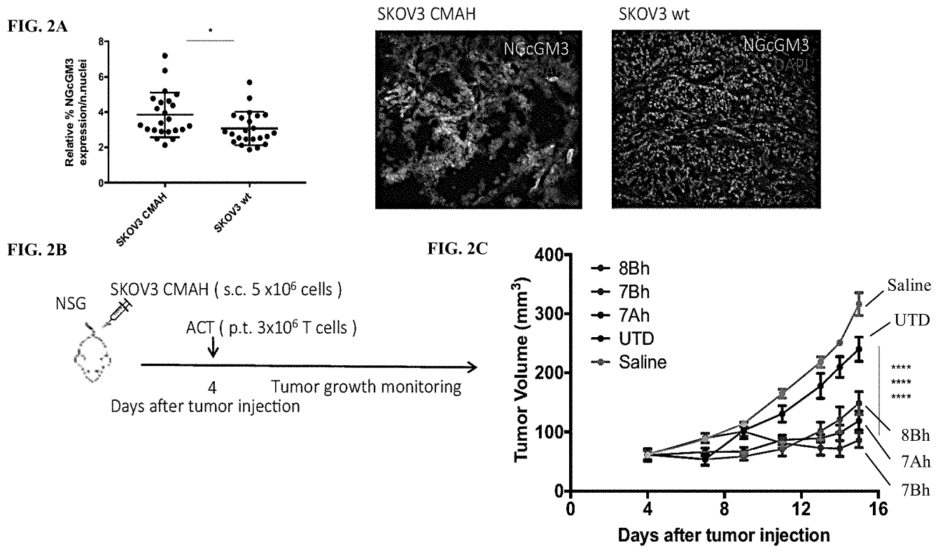

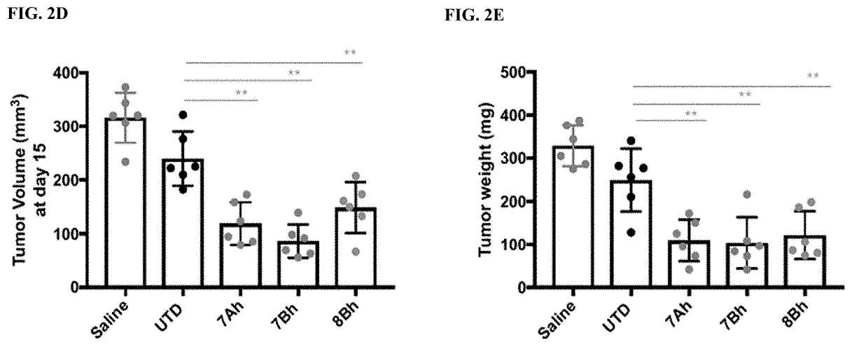

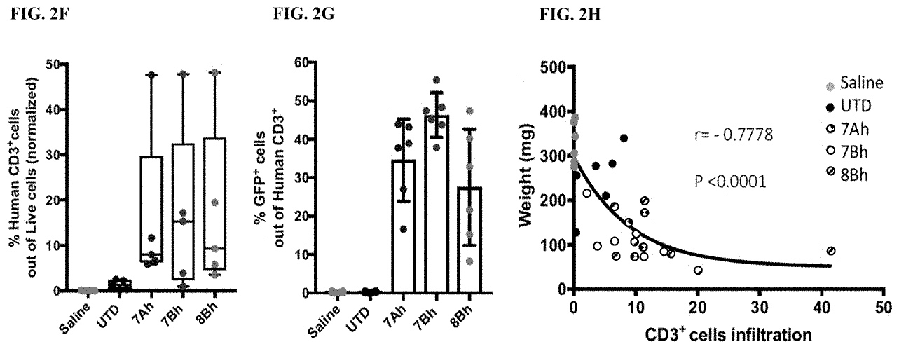

An ovarian SKOV3 cell line was obtained that is stably transduced to overexpress CMP-N-acetylneuraminic acid hydroxylase (SKOV3 CMAH cells). The SKOV3 CMAH cells generated NGcGM3 from NAcGM3 (FIG. 2A). To evaluate an in vivo therapeutic effect of the humanized 14F7-derived CAR T cells, NSG female mice were implemented with SKOV3 CMAH and, upon tumor establishment, 3×10⁶ 7 Ah, 7Bh, 8Bh cells or UTD were adoptively transferred by a single peritumoral injection individually to different mice (FIG. 3B). A saline solution was also used as a negative control. 7Ah, 7Bh, and 8Bh each equally controlled tumor growth, while UTD or saline had no effect. 7Bh demonstrated the highest target-cell killing efficiency of the of the humanized 14F7-derived CAR T cells (FIGS. 2C and 2D).

To further analyze a therapeutic effect of the of the three humanized 14F7-derived CAR T cells, upon euthanasia collected tumors were weighed and subjected to further analyses. The masses of the 7Ah, 7Bh and 8Bh cell-treated tumors were smaller than UTD and saline-treated tumors (p<0.01, Mann-Whitney u test) (FIG. 2E). Human T cells in the tumors were detected by staining with human anti-CD3 Ab (FIG. 2F). Also, CAR expression of the human T cells in the tumors was assessed by detecting GFP (FIG. 2G). An inverse correlation was demonstrated between weight of a tumor and humanized 14F7-derived CAR T cell presence within the tumor (r=−0.7778, p<0.0001) (FIG. 211).

Sequence Listing

SEQ ID NO: 1 VH
QVQLQQSGASMKMSCRASGYSFTSYWIHWLKQRPDQGLEWIGYIDPATAYTESNQKFK
DKAILTADRSSNTAFMYLNSLTSEDSAVYYCARESPRLRRGIYYYAMDYWGQGTSVTV
SS
SEQ ID NO: 2 VH FR1
QVQLQQSGASMKMSCRASGYSFT
SEQ ID NO: 3 VH CDR1
SYWIH
SEQ ID NO: 4 VH FR2
WLKQRPDQGLEWIG
SEQ ID NO: 5 VH CDR2
YIDPATAYTESNQKFKD
SEQ ID NO: 6 VH FR3
KAILTADRSSNTAFMYLNSLTSEDSAVYYCAR
SEQ ID NO: 7 VH CDR3
ESPRLRRGIYYYAMDY
SEQ ID NO: 8 VH FR4
WGQGTSVTVSS
SEQ ID NO: 9 VL 7Ah
QSVVTQPPSASGGPGQSLTISCTGTSSDVGGYNHVSWYQQHPGKAPKLMIYDVSKRPSG
VPHRFSGSKSGNTASLTVSGLQAEDEAVYYCSSYAGSNNLVFGGGTKVTVL
SEQ ID NO: 10 VL 7Bh
DIQMTQTPSSLSASVGDRVTITCRASQSISSFLNWYQQKPGKAPKLLIYAASNLQSGVPS
RFSGRGSGTDFTLTISSLQPEDFAAYYCQQGYTTPLTFGQGTKLE
SEQ ID NO: 11 VL 8Bh
SSELTQDPAVSVALGQTVRITCQGDSLRSYYASWYQQKPGQAPVLVIYGKNNRPSGIPD
RFSGSSSGNTASLTITGAQAEDEADYYCNSRDSSGNHVVFGGGTKLTVL
SEQ ID NO: 12 Core hinge domain
EPKSCDKTHTCPPCP
SEQ ID NO: 13 Core hinge domain
ERKCCVECPPCP
SEQ ID NO: 14 Core hinge domain
ELKTPLGDTTHTCPRCP(EPKSCDTPPPCPRCP)₃
SEQ ID NO: 15 Core hinge domain
ESKYGPPCPSCP
SEQ ID NO: 16 Linker
APQAKSSGSGSESKVD
SEQ ID NO: 17 7Ah FR1
QSVVTQPPSASGGPGQSLTISC
SEQ ID NO: 18 7Ah CDR1
TGTSSDVGGYNHVS
SEQ ID NO: 19 7Ah FR2
WYQQHPGKAPKLMIY
SEQ ID NO: 20 7Ah CDR2
DVSKRPS
SEQ ID NO: 21 7Ah FR3
GVPHRFSGSKSGNTASLTVSGLQAEDEAVYYC
SEQ ID NO: 22 7Ah CDR3
SSYAGSNNLVF
SEQ ID NO: 23 7Ah FR4
GGGTKVTVL
SEQ ID NO: 24 7Bh FR1
DIQMTQTPSSLSASVGDRVTITC
SEQ ID NO: 25 7Bh CDR1
RASQSISSFLN
SEQ ID NO: 26 7Bh FR2
WYQQKPGKAPKLLIY
SEQ ID NO: 27 7Bh CDR2
AASNLQS
SEQ ID NO: 28 7Bh FR3
GVPSRFSGRGSGTDFTLTISSLQPEDFAAYYC
SEQ ID NO: 29 7Bh CDR3
QQGYTTPLTF
SEQ ID NO: 30 7Bh FR4
GQGTKLE
SEQ ID NO: 31 8Bh FR1
SSELTQDPAVSVALGQTVRITC
SEQ ID NO: 32 8Bh CDR1
QGDSLRSYYAS
SEQ ID NO: 33 8Bh FR2
WYQQKPGQAPVLVIY
SEQ ID NO: 34 8Bh CDR2
GKNNRPS
SEQ ID NO: 35 8Bh FR3
GIPDRFSGSSSGNTASLTITGAQAEDEADYYC
SEQ ID NO: 36 8Bh CDR3
NSRDSSGNHVVF
SEQ ID NO: 37 8Bh FR4
GGGTKLTVL
SEQ ID NO: 38 Separation sequence
GSGEGRGSLLTCGDVEENPGP
SEQ ID NO: 39 Separation sequence
GSGSRVTELLYRMKRAETYCPRPLLAIHPTEARHKQKIVAPVKQLLNFDLLKLAGDVES
NPGP
SEQ ID NO: 40 Separation sequence
LLCFLLLLLSGDVELNPGP
SEQ ID NO: 41 Separation sequence
HHFMFLLLLLAGDIELNPGP
SEQ ID NO: 42 Separation sequence
WFLVLLSFILSGDIEVNPGP
SEQ ID NO: 43 Separation sequence
KNCAMYMLLLSGDVETNPGP
SEQ ID NO: 44 Separation sequence
MVISQLMLKLAGDVEENPGP
SEQ ID NO: 45 Separation sequence
GSGATNFSLLKQAGDVEENPGP
SEQ ID NO: 46 Separation sequence
GSGQCTNYALLKLAGDVESNPGP
SEQ ID NO: 47 PGK promoter
GGGTAGGGGAGGCGCTTTTCCCAAGGCAGTCTGGAGCATGCGCTTTAGCAGCCCCG
CTGGGCACTTGGCGCTACACAAGTGGCCTCTGGCCTCGCACACATTCCACATCCACC
GGTAGGCGCCAACCGGCTCCGTTCTTTGGTGGCCCCTTCGCGCCACCTTCTACTCCTC
CCCTAGTCAGGAAGTTCCCCCCCGCCCCGCAGCTCGCGTCGTGCAGGACGTGACAA
ATGGAAGTAGCACGTCTCACTAGTCTCGTGCAGATGGACAGCACCGCTGAGCAATG
GAAGCGGGTAGGCCTTTGGGGCAGCGGCCAATAGCAGCTTTGCTCCTTCGCTTTCTG
GGCTCAGAGGCTGGGAAGGGGTGGGTCCGGGGGCGGGCTCAGGGGCGGGCTCAGG
GGCGGGGCGGGCGCCCGAAGGTCCTCCGGAGGCCCGGCATTCTGCACGCTTCAAAA
GCGCACGTCTGCCGCGCTGTTCTCCTCTTCCTCATCTCCGGGCCTTTCG
SEQ ID NO: 48 IC
aggagtaagaggagcaggctcctgcacagtgactacatgaacatgactccccgccgccccgggcccacccgcaagcattaccagcccta
tgccccaccacgcgacttcgcagcctatcgctcc
SEQ ID NO: 49 TM
ttttgggtgctggtggtggttggtggagtcctggcttgctatagcttgctagtaacagtggcctttattattttctgggtg
SEQ ID NO: 50 Zeta signalling domain
agagtgaagttcagcaggagcgcagacgcccccgcgtaccagcagggccagaaccagctctataacgagctcaatctaggacgaagag
aggagtacgatgttttggacaagagacgtggccgggaccctgagatggggggaaagccgagaaggaagaaccctcaggaaggcctgta
caatgaactgcagaaagataagatggcggaggcctacagtgagattgggatgaaaggcgagcgccggaggggcaaggggcacgatgg
cctttaccagggtctcagtacagccaccaaggacacctacgacgcccttcacatgcaggccctgccccctcgctaa
SEQ ID NO: 51 Hinge
accacgacgccagcgccgcgaccaccaacaccggcgcccaccatcgcgtcgcagcccctgtccctgcgcccagaggcgtgccggcca
gcggcggggggcgcagtgcacacgagggggctggacttcg
SEQ ID NO: 52 TM
FWVLVVVGGVLACYSLLVTVAFIIFWV
SEQ ID NO: 53 Hinge
TTTPAPRPPTPAPTIASQPLSLRPEACRPAAGGAVHTRGLDFACD
SEQ ID NO: 54 IC
RSKRSRLLHSDYMNMTPRRPGPTRKHYQPYAPPRDFAAYRS
SEQ ID NO: 55 Zeta signalling domain
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 56 Leader Sequence
MALPVTALLLPLALLLHAARP
SEQ ID NO: 57 7Ah scFv
CAAGTCCAGCTGCAGCAGAGCGGCGCCAGCATGAAGATGAGCTGTAGAGCCAGCGG
CTACAGCTTCACCAGCTACTGGATCCACTGGCTGAAGCAGAGGCCAGATCAGGGCC
TCGAGTGGATCGGCTATATCGATCCTGCCACCGCCTACACCGAGAGCAACCAGAAG
TTCAAGGACAAGGCCATCCTGACCGCCGACAGAAGCAGCAACACCGCCTTCATGTA
CCTGAACAGCCTGACCAGCGAGGACAGCGCCGTGTACTATTGCGCCAGAGAGAGCC
CCAGACTGCGGAGAGGCATCTACTACTACGCCATGGACTATTGGGGCCAGGGCACC
AGCGTGACAGTTTCTTCTGCCCCTCAAGCCAAGAGCAGCGGCAGCGGATCTGAGTCT
AAGGTGGACCAGAGCGTGGTCACCCAGCCTCCATCTGCTAGCGGAGGACCTGGACA
GAGCCTGACAATCAGCTGTACCGGCACCAGCTCTGATGTCGGCGGCTACAATCACGT
GTCCTGGTATCAGCAGCACCCCGGCAAAGCCCCTAAGCTGATGATCTACGACGTGTC
CAAGAGGCCTAGCGGCGTGCCACACAGATTTTCCGGCAGCAAGTCTGGCAATACCG
CCTCTCTGACCGTGTCTGGACTGCAGGCCGAAGATGAGGCCGTGTATTACTGCAGCA
GCTACGCCGGCTCCAACAACCTGGTTTTTGGCGGAGGCACCAAAGTGACCGTGCTGT
SEQ ID NO: 58 7Bh CAR
atggccttaccagtgaccgccttgctcctgccgctggccttgctgctccacgccgccaggccgggatctCAAGTCCAGCTGCA
GCAGAGCGGCGCCAGCATGAAGATGAGCTGTAGAGCCAGCGGCTACAGCTTCACCA
GCTACTGGATCCACTGGCTGAAGCAGAGGCCAGATCAGGGCCTCGAGTGGATCGGC
TATATCGATCCTGCCACCGCCTACACCGAGAGCAACCAGAAGTTCAAGGACAAGGC
CATCCTGACCGCCGACAGAAGCAGCAACACCGCCTTCATGTACCTGAACAGCCTGA
CCAGCGAGGACAGCGCCGTGTACTATTGCGCCAGAGAGAGCCCCAGACTGCGGAGA
GGCATCTACTACTACGCCATGGACTATTGGGGCCAGGGCACCAGCGTGACAGTTTCT
TCTGCCCCTCAAGCCAAGAGCAGCGGCAGCGGATCTGAGAGCAAGGTGGACGACAT
CCAGATGACCCAGACACCTAGCAGCCTGAGCGCCTCTGTGGGCGACAGAGTGACCA
TCACATGCAGAGCCAGCCAGAGCATCAGCAGCTTTCTGAACTGGTATCAGCAGAAG
CCCGGCAAGGCCCCTAAACTGCTGATCTACGCCGCCAGCAATCTGCAGAGCGGAGT
GCCTAGCAGATTCAGCGGAAGAGGCTCCGGCACCGATTTCACCCTGACCATATCTAG
CCTGCAGCCAGAGGACTTCGCCGCCTACTATTGTCAGCAGGGCTACACCACACCTCT
GACCTTTGGCCAGGGGACCAAGCTGGAAGTctagcaccacgacgccagcgccgcgaccaccaacaccggc
gcccaccatcgcgtcgcagcccctgtccctgcgcccagaggcgtgccggccagcggcggggggcgcagtgcacacgagggggctgg
acttcgcctgtgatttttgggtgctggtggtggttggtggagtcctggcttgctatagcttgctagtaacagtggcctttattattttctgggt
gaggagtaagaggagcaggctcctgcacagtgactacatgaacatgactccccgccgccccgggcccacccgcaagcattaccagccctatg
ccccaccacgcgacttcgcagcctatcgctccatcgatagagtgaagttcagcaggagcgcagacgcccccgcgtaccagcagggcca
gaaccagctctataacgagctcaatctaggacgaagagaggagtacgatgttttggacaagagacgtggccgggaccctgagatggggg
gaaagccgagaaggaagaaccctcaggaaggcctgtacaatgaactgcagaaagataagatggcggaggcctacagtgagattgggat
gaaaggcgagcgccggaggggcaaggggcacgatggcctttaccagggtctcagtacagccaccaaggacacctacgacgcccttcac
atgcaggccctgccccctcgctaa
SEQ ID NO: 59 7Bh scFv
CAAGTCCAGCTGCAGCAGAGCGGCGCCAGCATGAAGATGAGCTGTAGAGCCAGCGG
CTACAGCTTCACCAGCTACTGGATCCACTGGCTGAAGCAGAGGCCAGATCAGGGCC
TCGAGTGGATCGGCTATATCGATCCTGCCACCGCCTACACCGAGAGCAACCAGAAG
TTCAAGGACAAGGCCATCCTGACCGCCGACAGAAGCAGCAACACCGCCTTCATGTA
CCTGAACAGCCTGACCAGCGAGGACAGCGCCGTGTACTATTGCGCCAGAGAGAGCC
CCAGACTGCGGAGAGGCATCTACTACTACGCCATGGACTATTGGGGCCAGGGCACC
AGCGTGACAGTTTCTTCTGCCCCTCAAGCCAAGAGCAGCGGCAGCGGATCTGAGAG
CAAGGTGGACGACATCCAGATGACCCAGACACCTAGCAGCCTGAGCGCCTCTGTGG
GCGACAGAGTGACCATCACATGCAGAGCCAGCCAGAGCATCAGCAGCTTTCTGAAC
TGGTATCAGCAGAAGCCCGGCAAGGCCCCTAAACTGCTGATCTACGCCGCCAGCAA
TCTGCAGAGCGGAGTGCCTAGCAGATTCAGCGGAAGAGGCTCCGGCACCGATTTCA
CCCTGACCATATCTAGCCTGCAGCCAGAGGACTTCGCCGCCTACTATTGTCAGCAGG
GCTACACCACACCTCTGACCTTTGGCCAGGGGACCAAGCTGGAAGT
SEQ ID NO: 60 8Bh CAR
atggccttaccagtgaccgccttgctcctgccgctggccttgctgctccacgccgccaggccgCAAGTCCAGCTGCAGCA
GAGCGGCGCCAGCATGAAGATGAGCTGTAGAGCCAGCGGCTACAGCTTCACCAGCT
ACTGGATCCACTGGCTGAAGCAGAGGCCAGATCAGGGCCTCGAGTGGATCGGCTAT
ATCGATCCTGCCACCGCCTACACCGAGAGCAACCAGAAGTTCAAGGACAAGGCCAT
CCTGACCGCCGACAGAAGCAGCAACACCGCCTTCATGTACCTGAACAGCCTGACCA
GCGAGGACAGCGCCGTGTACTATTGCGCCAGAGAGAGCCCCAGACTGCGGAGAGGC
ATCTACTACTACGCCATGGACTATTGGGGCCAGGGCACCAGCGTGACAGTTTCTTCT
GCCCCTCAAGCCAAGAGCAGCGGCAGCGGATCTGAGAGCAAAGTGGATAGCAGCG
AGCTGACACAGGACCCCGCTGTGTCTGTTGCTCTGGGCCAGACAGTGCGGATTACCT
GTCAGGGCGATAGCCTGCGGAGCTACTATGCCAGCTGGTATCAGCAGAAGCCCGGA
CAGGCTCCTGTGCTGGTCATCTACGGCAAGAACAACAGGCCCAGCGGCATCCCCGA
TAGATTTTCTGGCAGCAGCTCCGGCAATACCGCCAGCCTGACAATTACTGGCGCCCA
GGCCGAAGATGAGGCCGACTACTACTGCAACAGCAGAGACTCCAGCGGCAATCACG
TGGTGTTTGGCGGCGGAACAAAGCTGACAGTGCTGTctagcaccacgacgccagcgccgcgaccacc
aacaccggcgcccaccatcgcgtcgcagcccctgtccctgcgcccagaggcgtgccggccagcggcggggggcgcagtgcacacga
gggggctggacttcgcctgtgatttttgggtgctggtggtggttggtggagtcctggcttgctatagcttgctagtaacagtggcctttattat
tttctgggtgaggagtaagaggagcaggctcctgcacagtgactacatgaacatgactccccgccgccccgggcccacccgcaagcattacc
agccctatgccccaccacgcgacttcgcagcctatcgctccatcgatagagtgaagttcagcaggagcgcagacgcccccgcgtaccag
cagggccagaaccagctctataacgagctcaatctaggacgaagagaggagtacgatgttttggacaagagacgtggccgggaccctga
gatggggggaaagccgagaaggaagaaccctcaggaaggcctgtacaatgaactgcagaaagataagatggcggaggcctacagtga
gattgggatgaaaggcgagcgccggaggggcaaggggcacgatggcctttaccagggtctcagtacagccaccaaggacacctacgac
gcccttcacatgcaggccctgccccctcgctaa
SEQ ID NO: 61 7Ah CAR
MALPVTALLLPLALLLHAARPGSQVQLQQSGASMKMSCRASGYSFTSYWIHWLKQRPD
QGLEWIGYIDPATAYTESNQKFKDKAILTADRSSNTAFMYLNSLTSEDSAVYYCARESP
RLRRGIYYYAMDYWGQGTSVTVSSAPQAKSSGSGSESKVDQSVVTQPPSASGGPGQSL
TISCTGTSSDVGGYNHVSWYQQHPGKAPKLMIYDVSKRPSGVPHRFSGSKSGNTASLTV
SGLQAEDEAVYYCSSYAGSNNLVFGGGTKVTVLASTTTPAPRPPTPAPTIASQPLSLRPE
ACRPAAGGAVHTRGLDFACDFWVLVVVGGVLACYSLLVTVAFIIFWVRSKRSRLLHSD
YMNMTPRRPGPTRKHYQPYAPPRDFAAYRSIDRVKFSRSADAPAYQQGQNQLYNELNL
GRREEYDVLDKRRGRDPEMGGKPRRKNPQEGLYNELQKDKMAEAYSEIGMKGERRRG
KGHDGLYQGLSTATKDTYDALHMQALPPR
SEQ ID NO: 62 7Bh CAR
MALPVTALLLPLALLLHAARPGSQVQLQQSGASMKMSCRASGYSFTSYWIHWLKQRPD
QGLEWIGYIDPATAYTESNQKFKDKAILTADRSSNTAFMYLNSLTSEDSAVYYCARESP
RLRRGIYYYAMDYWGQGTSVTVSSAPQAKSSGSGSESKVDDIQMTQTPSSLSASVGDR
VTITCRASQSISSFLNWYQQKPGKAPKLLIYAASNLQSGVPSRFSGRGSGTDFTLTISSLQ
PEDFAAYYCQQGYTTPLTFGQGTKLEASTTTPAPRPPTPAPTIASQPLSLRPEACRPAAGG
AVHTRGLDFACDFWVLVVVGGVLACYSLLVTVAFIIFWVRSKRSRLLHSDYMNMTPRR
PGPTRKHYQPYAPPRDFAAYRSIDRVKFSRSADAPAYQQGQNQLYNELNLGRREEYDV
LDKRRGRDPEMGGKPRRKNPQEGLYNELQKDKMAEAYSEIGMKGERRRGKGHDGLY
QGLSTATKDTYDALHMQALPPR
SEQ ID NO: 63 8Bh CAR
MALPVTALLLPLALLLHAARPGSQVQLQQSGASMKMSCRASGYSFTSYWIHWLKQRPD
QGLEWIGYIDPATAYTESNQKFKDKAILTADRSSNTAFMYLNSLTSEDSAVYYCARESP
RLRRGIYYYAMDYWGQGTSVTVSSAPQAKSSGSGSESKVDSSELTQDPAVSVALGQTV
RITCQGDSLRSYYASWYQQKPGQAPVLVIYGKNNRPSGIPDRFSGSSSGNTASLTITGAQ
AEDEADYYCNSRDSSGNHVVFGGGTKLTVLASTTTPAPRPPTPAPTIASQPLSLRPEACR
PAAGGAVHTRGLDFACDFWVLVVVGGVLACYSLLVTVAFIIFWVRSKRSRLLHSDYMN
MTPRRPGPTRKHYQPYAPPRDFAAYRSIDRVKFSRSADAPAYQQGQNQLYNELNLGRR
EEYDVLDKRRGRDPEMGGKPRRKNPQEGLYNELQKDKMAEAYSEIGMKGERRRGKGH
DGLYQGLSTATKDTYDALHMQALPPR
SEQ ID NO: 64 7Ah CAR
atggccttaccagtgaccgccttgctcctgccgctggccttgctgctccacgccgccaggccgggatctCAAGTCCAGCTGCA
GCAGAGCGGCGCCAGCATGAAGATGAGCTGTAGAGCCAGCGGCTACAGCTTCACCA
GCTACTGGATCCACTGGCTGAAGCAGAGGCCAGATCAGGGCCTCGAGTGGATCGGC
TATATCGATCCTGCCACCGCCTACACCGAGAGCAACCAGAAGTTCAAGGACAAGGC
CATCCTGACCGCCGACAGAAGCAGCAACACCGCCTTCATGTACCTGAACAGCCTGA
CCAGCGAGGACAGCGCCGTGTACTATTGCGCCAGAGAGAGCCCCAGACTGCGGAGA
GGCATCTACTACTACGCCATGGACTATTGGGGCCAGGGCACCAGCGTGACAGTTTCT
TCTGCCCCTCAAGCCAAGAGCAGCGGCAGCGGATCTGAGTCTAAGGTGGACCAGAG
CGTGGTCACCCAGCCTCCATCTGCTAGCGGAGGACCTGGACAGAGCCTGACAATCA
GCTGTACCGGCACCAGCTCTGATGTCGGCGGCTACAATCACGTGTCCTGGTATCAGC
AGCACCCCGGCAAAGCCCCTAAGCTGATGATCTACGACGTGTCCAAGAGGCCTAGC
GGCGTGCCACACAGATTTTCCGGCAGCAAGTCTGGCAATACCGCCTCTCTGACCGTG
TCTGGACTGCAGGCCGAAGATGAGGCCGTGTATTACTGCAGCAGCTACGCCGGCTC
CAACAACCTGGTTTTTGGCGGAGGCACCAAAGTGACCGTGCTGTctagcaccacgacgccagc
gccgcgaccaccaacaccggcgcccaccatcgcgtcgcagcccctgtccctgcgcccagaggcgtgccggccagcggcggggggcg
cagtgcacacgagggggctggacttcgcctgtgatttttgggtgctggtggtggttggtggagtcctggcttgctatagcttgctagtaacagt
ggcctttattattttctgggtgaggagtaagaggagcaggctcctgcacagtgactacatgaacatgactccccgccgccccgggcccacc
cgcaagcattaccagccctatgccccaccacgcgacttcgcagcctatcgctccatcgatagagtgaagttcagcaggagcgcagacgcc
cccgcgtaccagcagggccagaaccagctctataacgagctcaatctaggacgaagagaggagtacgatgttttggacaagagacgtgg
ccgggaccctgagatggggggaaagccgagaaggaagaaccctcaggaaggcctgtacaatgaactgcagaaagataagatggcgga
ggcctacagtgagattgggatgaaaggcgagcgccggaggggcaaggggcacgatggcctttaccagggtctcagtacagccaccaag
gacacctacgacgcccttcacatgcaggccctgccccctcgctaa
SEQ ID NO: 65 Separation sequence
EGRGSLLTCGDVEENPGP
SEQ ID NO: 66 8Bh scFv
CAAGTCCAGCTGCAGCAGAGCGGCGCCAGCATGAAGATGAGCTGTAGAGCCAGCGG
CTACAGCTTCACCAGCTACTGGATCCACTGGCTGAAGCAGAGGCCAGATCAGGGCC
TCGAGTGGATCGGCTATATCGATCCTGCCACCGCCTACACCGAGAGCAACCAGAAG
TTCAAGGACAAGGCCATCCTGACCGCCGACAGAAGCAGCAACACCGCCTTCATGTA
CCTGAACAGCCTGACCAGCGAGGACAGCGCCGTGTACTATTGCGCCAGAGAGAGCC
CCAGACTGCGGAGAGGCATCTACTACTACGCCATGGACTATTGGGGCCAGGGCACC
AGCGTGACAGTTTCTTCTGCCCCTCAAGCCAAGAGCAGCGGCAGCGGATCTGAGAG
CAAAGTGGATAGCAGCGAGCTGACACAGGACCCCGCTGTGTCTGTTGCTCTGGGCC
AGACAGTGCGGATTACCTGTCAGGGCGATAGCCTGCGGAGCTACTATGCCAGCTGG
TATCAGCAGAAGCCCGGACAGGCTCCTGTGCTGGTCATCTACGGCAAGAACAACAG
GCCCAGCGGCATCCCCGATAGATTTTCTGGCAGCAGCTCCGGCAATACCGCCAGCCT
GACAATTACTGGCGCCCAGGCCGAAGATGAGGCCGACTACTACTGCAACAGCAGAG
ACTCCAGCGGCAATCACGTGGTGTTTGGCGGCGGAACAAAGCTGACAGTGCTGT
SEQ ID NO: 67 7Ah scFv
QVQLQQSGASMKMSCRASGYSFTSYWIHWLKQRPDQGLEWIGYIDPATAYTESNQKFK
DKAILTADRSSNTAFMYLNSLTSEDSAVYYCARESPRLRRGIYYYAMDYWGQGTSVTV
SSAPQAKSSGSGSESKVDQSVVTQPPSASGGPGQSLTISCTGTSSDVGGYNHVSWYQQH
PGKAPKLMIYDVSKRPSGVPHRFSGSKSGNTASLTVSGLQAEDEAVYYCSSYAGSNNLV
FGGGTKVTVL
SEQ ID NO: 68 7Bh scFv
QVQLQQSGASMKMSCRASGYSFTSYWIHWLKQRPDQGLEWIGYIDPATAYTESNQKFK
DKAILTADRSSNTAFMYLNSLTSEDSAVYYCARESPRLRRGIYYYAMDYWGQGTSVTV
SSAPQAKSSGSGSESKVDDIQMTQTPSSLSASVGDRVTITCRASQSISSFLNWYQQKPGK
APKLLIYAASNLQSGVPSRFSGRGSGTDFTLTISSLQPEDFAAYYCQQGYTTPLTFGQGT
KLE
SEQ ID NO: 69 8Bh scFv
QVQLQQSGASMKMSCRASGYSFTSYWIHWLKQRPDQGLEWIGYIDPATAYTESNQKFK
DKAILTADRSSNTAFMYLNSLTSEDSAVYYCARESPRLRRGIYYYAMDYWGQGTSVTV
SSAPQAKSSGSGSESKVDSSELTQDPAVSVALGQTVRITCQGDSLRSYYASWYQQKPGQ
APVLVIYGKNNRPSGIPDRFSGSSSGNTASLTITGAQAEDEADYYCNSRDSSGNHVVFGG
GTKLTVL
SEQ ID NO: 70 Leader Sequence
atggccttaccagtgaccgccttgctcctgccgctggccttgctgctccacgccgccaggccg
SEQ ID NO: 71 Leader Sequence 2
ATGGACTGGATCTGGCGCATCCTGTTTCTCGTGGGAGCCGCCACAGGCGCCCATTCT
SEQ ID NO: 72 Leader Sequence 2
MDWIWRILFLVGAATGAHS
SEQ ID NO: 73 Leader Sequence 3
MNRGVPFRHLLLVLQLALLPAATQG
SEQ ID NO: 74 2A consensus sequence
D-X-E-X-NPGP (X is any amino acid)
SEQ ID NO: 75
(GGGS)n (n is a positive integer equal to or greater than 1)
SEQ ID NO: 76
GGGGS GGGGS GGGGS GGGGS
SEQ ID NO: 77
GGGGS GGGGS GGGGS
SEQ ID NO: 78
GGGS
SEQ ID NO: 79
GSTSGSGKPGSGEGSTKG

### REFERENCES

1. Brentjens R J, Davila M L, Riviere I et al. CD19-targeted T cells rapidly induce molecular remissions in adults with chemotherapy-refractory acute lymphoblastic leukemia. Sci Transl Med 2013; 5: 177ra138.
2. Maude S L, Frey N, Shaw P A et al. Chimeric antigen receptor T cells for sustained remissions in leukemia. N Engl J Med 2014; 371: 1507-1517.
3. Maus M V, Grupp S A, Porter D L, June C H. Antibody-modified T cells: CARs take the front seat for hematologic malignancies. Blood 2014; 123: 2625-2635.
4. Morgan R A, Yang J C, Kitano M et al. Case report of a serious adverse event following the administration of T cells transduced with a chimeric antigen receptor recognizing ERBB2. Mol Ther 2010; 18: 843-851.
5. Varki, A. & Lowe, J. B. Biological Roles of Glycans. Essentials of Glycobiology (Cold Spring Harbor Laboratory Press, 2009).
6. Bull, C., den Brok, M. H. & Adema, G. J. Sweet escape: Sialic acids in tumor immune evasion. Biochim. Biophys. Acta-Rev. Cancer 1846, 238-246 (2014).
7. Rodriguez, E., Schetters, S. T. T. & Van Kooyk, Y. The tumour glyco-code as a novel immune checkpoint for immunotherapy. Nat. Rev. Immunol. 18, 204-211(2018).
8. Varki, A. & Schauer, R. Sialic Acids. Essentials of Glycobiology (Cold Spring Harbor Laboratory Press, 2009).
9. Varki, A. N-glycolylneuraminic acid deficiency in humans. Biochimie 83, (2001).
10. Oliva, J. P. et al. Clinical evidences of GM3 (NeuGc) ganglioside expression in human breast cancer using the 14F7 monoclonal antibody labelled with 99mTc. Breast Cancer Res. Treat. 96, 115-121 (2006).
11. Marquina, G. et al. Gangliosides expressed in human breast cancer. Cancer Res. 56, 5165-71 (1996).
12. Blanco, R. et al. Immunoreactivity of the 14F7 Mab Raised against N-Glycolyl GM3 Ganglioside in Epithelial Malignant Tumors from Digestive System. ISRN Gastroenterol. 2011, 645641 (2011).
13. Blanco, R. et al. Immunorecognition of the 14F7 Mab Raised against N-Glycolyl GM3 Ganglioside in Some Normal and Malignant Tissues from Genitourinary System. ISRN Pathol. 2011, 1-10 (2011).
14. Torbidoni, A. V. et al. Immunoreactivity of the 14F7 Mab raised against N-Glycolyl GM3 Ganglioside in retinoblastoma tumours. Acta Ophthalmol. 93, e294-e300 (2015).
15. Blanco, R. et al. Prognostic Significance of N-Glycolyl GM3 Ganglioside Expression in Non-Small Cell Lung Carcinoma Patients: New Evidences. Patholog. Res. Int. 2015, 132326 (2015).
16. Hedlund, M. et al. N-glycolylneuraminic acid deficiency in mice: implications for human biology and evolution. Mol. Cell. Biol. 27, 4340-6 (2007).
17. Yin, J. et al. Hypoxic culture induces expression of sialin, a sialic acid transporter, and cancer-associated gangliosides containing non-human sialic acid on human cancer cells. Cancer Res. 66, 2937-45 (2006).
18. Yin, J., Miyazaki, K., Shaner, R. L., Merrill, A. H. & Kannagi, R. Altered sphingolipid metabolism induced by tumor hypoxia—New vistas in glycolipid tumor markers. FEBS Lett. 584, 1872-1878 (2010).
19. Taylor, R. E. et al. Novel mechanism for the generation of human xeno-autoantibodies against the nonhuman sialic acid N-glycolylneuraminic acid. J. Exp. Med. 207, 1637-46 (2010).
20. de Leon, J. et al. Differential influence of the tumour-specific non-human sialic acid containing GM3 ganglioside on CD4+CD25− effector and naturally occurring CD4+CD25+ regulatory T cells function. Int. Immunol. 20, 591-600 (2008).
21. Vazquez, A. M. et al. Syngeneic Anti-Idiotypic Monoclonal Antibodies to an Anti-NeuGc-Containing Ganglioside Monoclonal Antibody. 17, 527-534 (1998).
22. Alfonso, M. et al. An Anti-Idiotype Vaccine Elicits a Specific Response to N-Glycolyl Sialic Acid Residues of Glycoconjugates in Melanoma Patients. J. Immunol. 168, 2523-2529 (2002).
23. Diaz, A. et al. Immune responses in breast cancer patients immunized with an anti-idiotype antibody mimicking NeuGc-containing gangliosides. Clin. Immunol. 107, 80-89 (2003).
24. Alfonso, S. et al. 1E10 anti-idiotype vaccine in non-small cell lung cancer: Experience in stage IIIb/IV patients. Cancer Biol. Ther. 6, 1847-1852 (2007).
25. Neninger, E. et al. Active immunotherapy with 1E10 anti-idiotype vaccine in patients with small cell lung cancer: Report of a phase I trial. Cancer Biol. Ther. 6, 145-150 (2007).
26. Labrada, M. et al. GM3(Neu5Gc) ganglioside: an evolution fixed neoantigen for cancer immunotherapy. Semin. Oncol. (2018). doi:10.1053/j.seminoncol.2018.04.003.
27. Carr, A. et al. A Mouse IgG1 Monoclonal Antibody Specific for N-Glycolyl GM3 Ganglioside Recognized Breast and Melanoma Tumors. HYBRIDOMA 19, (Mary Ann Liebert, Inc, 2000).
28. Krengel, U. et al. Structure and Molecular Interactions of a Unique Antitumor Antibody Specific for N-Glycolyl GM3. J. Biol. Chem. 279, 5597-5603 (2004).
29. Bjerregaard-Andersen, K. et al. Crystal structure of an L chain optimised 14F7 anti-ganglioside Fv suggests a unique tumour-specificity through an unusual H-chain CDR3 architecture. Sci. Rep. 8, 1-11 (2018).
30. Roque-Navarro, L. et al. Anti-ganglioside antibody-induced tumor cell death by loss of membrane integrity. Mol Cancer Ther 7, 2033-2074 (2008).
31. Blanco, R. et al. Prognostic Significance of N-Glycolyl GM3 Ganglioside Expression in Non-Small Cell Lung Carcinoma Patients: New Evidences. Patholog. Res. Int. 2015, 132326 (2015).
32. Lahera, T. et al. Prognostic role of 14F7 mab immunoreactivity against N-glycolyl GM3 ganglioside in colon cancer. J. Oncol. 2014, (2014).
33. Gargett, T. et al. GD2-specific CAR T Cells Undergo Potent Activation and Deletion Following Antigen Encounter but can be Protected from Activation-induced Cell Death by PD-1 Blockade. Mol. Ther. 24, 1135-1149 (2016).
34. Yu, J. et al. Anti-GD2/4-1BB chimeric antigen receptor T cell therapy for the treatment of Chinese melanoma patients. J. Hematol. Oncol. 11, 1(2018).
35. Mount, C. W. et al. Potent antitumor efficacy of anti-GD2 CART cells in H3-K27M+ diffuse midline gliomas. Nat. Med. 24, 572-579 (2018).
36. Gargett, T. et al. GD2-specific CAR T Cells Undergo Potent Activation and Deletion Following Antigen Encounter but can be Protected from Activation-induced Cell Death by PD-1 Blockade. Mol. Ther. 24, 1135-1149 (2016).
37. Yu, J. et al. Anti-GD2/4-1BB chimeric antigen receptor T cell therapy for the treatment of Chinese melanoma patients. J. Hematol. Oncol. 11, 1(2018).
38. Rojas, G. et al. Light-chain shuffling results in successful phage display selection of functional prokaryotic-expressed antibody fragments to N-glycolyl GM3 ganglioside. J. Immunol. Methods 293, 71-83 (2004).
39. Labrada, M. et al. GM3(Neu5Gc) ganglioside: an evolution fixed neoantigen for cancer immunotherapy. Semin. Oncol. 45, 41-51 (2018).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. An isolated nucleic acid molecule encoding a chimeric antigen receptor (CAR), wherein said CAR comprises an anti-NGcGM3 binding domain, a transmembrane domain, and an endodomain.

2. The isolated nucleic acid molecule of claim 1, wherein said anti-NGcGM3 binding domain comprises an anti-NGcGM3 heavy chain variable domain sequence comprising: a heavy chain complementary determining region 1 (HC CDR1) sequence SWIH (SEQ ID NO:3), a heavy chain complementary determining region 2 (HC CDR2) sequence YIDPATAYTESNQKFKD (SEQ ID NO:5), and a heavy chain complementary determining region 3 (HC CDR3) sequence ESPRLRRGIYYYAMDY (SEQ ID NO:7).

3.-7. (canceled)

8. The isolated nucleic acid molecule of claim 1, wherein the anti-NGcGM3 binding domain comprises an anti-NGcGM3 light chain variable domain amino acid sequence comprising:
(i) a light chain complementary determining region 1 (LC CDR1) sequence TGTSSDVGGYNHVS (SEQ ID NO:18), a light chain complementary determining region 2 (LC CDR2) sequence DVSKRPS (SEQ ID NO:20), and a light chain complementary determining region 3 (LC CDR3) sequence SSYAGSNNLVF (SEQ ID NO:22),
(ii) a light chain complementary determining region 1 (LC CDR1) sequence RASQSISSFLN (SEQ ID NO:25), a light chain complementary determining region 2 (LC CDR2) sequence AASNLQS (SEQ ID NO:27), and a light chain complementary determining region 3 (LC CDR3) sequence QQGYTTPLTF (SEQ ID NO:29), or
(iii) a light chain complementary determining region 1 (LC CDR1) sequence QGDSLRSYYAS (SEQ ID NO:32), a light chain complementary determining region 2 (LC CDR2) sequence GKNNRPS (SEQ ID NO:34), and a light chain complementary determining region 3 (LC CDR3) sequence NSRDSSGNHVVF (SEQ ID NO:36).

9. (canceled)

10. The isolated nucleic acid molecule of claim 1, wherein said anti-NGcGM3 binding domain comprises an anti-NGcGM3 light chain variable domain amino acid sequence of SEQ ID NO:9, SEQ ID NO:10, or SEQ ID NO:11 or a sequence with at least 80% identity to SEQ ID NO:9, SEQ ID NO:10, or SEQ ID NO:11.

11.-36. (canceled)

37. An isolated chimeric antigen receptor (CAR) molecule, wherein said CAR comprises an anti-NGcGM3 binding domain, a transmembrane domain, and an endodomain.

38. The isolated CAR molecule of claim 37, wherein said anti-NGcGM3 binding domain comprises an anti-NGcGM3 heavy chain variable domain sequence comprising: a heavy chain complementary determining region 1 (HC CDR1) sequence SYWIH (SEQ ID NO:3), a heavy chain complementary determining region 2 (HC CDR2) sequence YIDPATAYTESNQKFKD (SEQ ID NO:5), and a heavy chain complementary determining region 3 (HC CDR3) sequence ESPRLRRGIYYYAMDY (SEQ ID NO:7).

39.-43. (canceled)

44. The isolated CAR molecule of claim 37, wherein said anti-NGcGM3 binding domain comprises an anti-NGcGM3 light chain variable domain sequence comprising:
(i) a light chain complementary determining region 1 (LC CDR1) sequence TGTSSDVGGYNHVS (SEQ ID NO:18), a light chain complementary determining region 2 (LC CDR2) sequence DVSKRPS (SEQ ID NO:20), and a light chain complementary determining region 3 (LC CDR3) sequence SSYAGSNNLVF (SEQ ID NO:22), or
(ii) a light chain complementary determining region 1 (LC CDR1) sequence RASQSISSFLN (SEQ ID NO:25), a light chain complementary determining region 2 (LC CDR2) sequence AASNLQS (SEQ ID NO:27), and a light chain complementary determining region 3 (LC CDR3) sequence QQGYTTPLTF (SEQ ID NO:29), or
(iii) a light chain complementary determining region 1 (LC CDR1) sequence QGDSLRSYYAS (SEQ ID NO:32), a light chain complementary determining region 2 (LC CDR2) sequence GKNNRPS (SEQ ID NO:34), and a light chain complementary determining region 3 (LC CDR3) sequence NSRDSSGNHVVF (SEQ ID NO:36).

45. (canceled)

46. The isolated CAR molecule of claim 37, wherein said anti-NGcGM3 binding domain comprises an anti-NGcGM3 light chain variable domain amino acid sequence of SEQ ID NO:9, SEQ ID NO:10, or SEQ ID NO:11 or a sequence with at least 80% identity to SEQ ID NO:9, SEQ ID NO:10, or SEQ ID NO:11.

47. (canceled)

48. (canceled)

49. The isolated CAR molecule of claim 37, wherein said anti-NGcGM3 binding domain comprises a linker between a heavy chain variable domain and a light chain variable domain.

50. (canceled)

51. The isolated CAR molecule of claim 37, wherein a nucleotide sequence encoding said anti-NGcGM3 binding domain comprises SEQ ID NO:57, SEQ ID NO:59, or SEQ ID NO:66 or a sequence with at least 80% identity to SEQ ID NO:57, SEQ ID NO:59, or SEQ ID NO:66.

52. (canceled)

53. (canceled)

54. The isolated CAR molecule of claim 37, anti-NGcGM3 binding domain comprises an amino acid sequence of SEQ ID NO:67, SEQ ID NO:68, SEQ ID NO:69, or a sequence with at least 80% identity to SEQ ID NO:67, SEQ ID NO:68, or SEQ ID NO:69.

55. The isolated CAR molecule of claim 37, wherein the CAR includes a transmembrane domain derived from the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 or CD154.

56.-60. (canceled)

61. The isolated CAR molecule of claim 37, wherein the endodomain comprises an intracellular (IC) domain comprising a sequence derived from DAP10, DAP12, Fc epsilon receptor I gamma chain (FCER1G), FcR beta CD3-delta, CD3-epsilon, CD3-gamma, CD3-zeta, CD226, CD66d, CD79A, or CD79B.

62. (canceled)

63. (canceled)

64. The isolated CAR molecule of claim 37, wherein the endodomain comprises a signalling domain that comprises a sequence derived from DAP10, DAP12, Fc epsilon receptor I gamma chain (FCER1G), FcR beta CD3-delta, CD3-epsilon, CD3-gamma, CD3-zeta, CD226, CD66d, CD79A, or CD79B.

65.-69. (canceled)

70. A vector comprising a nucleic acid molecule encoding a CAR of claim 37.

71.-78. (canceled)

79. A cell comprising the vector of claim 70.

80.-82. (canceled)

83. A method of making a cell of claim 79.

84. A method of providing an anti-tumor immunity in a mammal comprising administering to the mammal an effective amount of a cell expressing the CAR molecule of claim 37.

85.-87. (canceled)

88. A method of treating a mammal having a disease associated with expression of NGcGM3 comprising administering to the mammal an effective amount of cells expressing a CAR molecule of claim 37.

89.-93. (canceled)

94. The isolated CAR molecule of claim 37 comprising an amino acid sequence of SEQ ID NO:58, SEQ ID NO:60, SEQ ID NO:61, SEQ ID NO:62, SEQ ID NO:63, SEQ ID NO:64 or a sequence with at least 80% identity to SEQ ID NO:58, SEQ ID NO:60, SEQ ID NO:61, SEQ ID NO:62, SEQ ID NO:63, SEQ ID NO:64 thereof.

95.-99. (canceled)
